SEQUENCE LISTING (7 sequences)

SEQ ID NO: 1 - protein, 5 aa, Artificial Sequence
  recombinant bovine somatotropin
  MOD_RES (1)..(1): AMIDATION
MDQFP

SEQ ID NO: 2 - protein, 12 aa, Artificial Sequence
  recombinant bovine somatotropin
  MOD_RES (1)..(1): AMIDATION
MFPLDDDDDKFP

SEQ ID NO: 3 - protein, 11 aa, Artificial Sequence
  recombinant bovine somatotropin
  MOD_RES (1)..(1): AMIDATION
MFPLDDDDKFP

SEQ ID NO: 4 - protein, 17 aa, Artificial Sequence
  recombinant bovine somatotropin
MFPAMSLSGLFANAVLR

SEQ ID NO: 5 - protein, 17 aa, Artificial Sequence
  recombinant bovine somatotropin
AFPAMSLSGLFANAVLR

SEQ ID NO: 6 - protein, 26 aa, Bos taurus
MMAAGPRTSLLLAFALLCLPWTQVVG

SEQ ID NO: 7 - protein, 191 aa, Bos taurus
AFPAMSLSGLFANAVLRAQHLHQLAADTFKEFERTYIPEGQRYSIQNTQVAFCFSETIPAPTGKNEAQQKSDLELLRISLLLIQSWLGPLQFLSRVFTNSLVFGTSDRVYEKLKDLEEGILALMRELEDGTPRAGQILKQTYDKFDTNMRSDDALLKNYGLLSCFRKDLHKTETYLRVMKCRRFGEASCAF

(19) United States
(12) Patent Application Publication
(10) Pub. No.: US 2017/0354717 A1
(43) Pub. Date: Dec. 14, 2017

(54) METHODS AND MEANS FOR DETERMINING TREATMENT OF SUBJECTS WITH EXOGENOUS SOMATOTROPIN

(71) Applicant: Stichting Wageningen Research, Wageningen (NL)

(72) Inventors: Nathalie Gabriëlle Esther Smits, Ede (NL); Marco Hendrik Blokland, De Meern (NL); Michel Wilhelmus Franciscus Nielen, Arnhem (NL)

(73) Assignee: Stichting Wageningen Research, Wageningen (NL)

(21) Appl. No.: 15/619,186

(22) Filed: Jun. 9, 2017

Related U.S. Application Data
(60) Provisional application No. 62/347,660, filed on Jun. 9, 2016.

(51) Int. Cl. A61K 38/27; C07K 14/575; C07K 14/61; C12N 15/09; C07K 14/00

(52) U.S. Cl. CPC A61K 38/27; C07K 14/61; C12N 15/09; C07K 14/575; C07K 14/00

(57) ABSTRACT

Described are methods for determining whether a subject has been treated with exogenous somatotropin. The disclosure further relates to kits for determining whether a subject has been treated with exogenous somatotropin, and to the use of such kits for determining whether a subject has been treated with exogenous somatotropin.

### PRIORITY CLAIM

This application claims the benefit of the filing date of U.S. Provisional Patent Application Ser. No. 62/347,660, filed Jun. 9, 2016, the contents of the entirety of which are incorporated herein by this reference.

### TECHNICAL FIELD

The application relates to the identification of a sample that is obtained from a subject, preferably of a bovine, more preferably of a cow, that has been treated with exogenous somatotropin, preferably with recombinant bovine somatotropin. Preferably, the sample is derived from milk or from blood, preferably from serum.

### BACKGROUND

1 Recombinant bovine somatotropin (rbST) is a synthetic duplicate or a slightly modified duplicate of bovine growth hormone (BGH), also termed bovine somatotropin (bST). BGH can be found naturally in milk. The synthetic hormone is periodically injected into cows and makes the mammary glands of dairy cows take in more nutrients from the bloodstream. Administration of rbST to dairy cows improves the efficiency of milk synthesis and increases milk production by 10%-40%.

Since 1985, rbST has led to concerns regarding human health, animal welfare, and environmental and economic impacts. There are no biological side effects that have been reported for humans. However, an increase in insulin-like growth factor (IGF-1) in milk from cattle that was treated with rbST has been reported. Increased levels of IGF-1 in turn may prevent a cancerous cell from going into apoptosis and have been linked to colon and breast cancer. In addition, cattle that was treated with rbST have an increased risk of developing mastitis; as a consequence, cattle subjected to rbST is often treated with antibiotics, of which remnants are also present in milk made available for human consumption.

At present, cattle that were treated with rbST cannot be discriminated from cattle that were not treated with recombinant hormone. In addition, pasteurization destroys approximately 90% of bST and rbST that is present in milk, and there are no tests available to identify a sample, for example, blood or milk, from cattle that were treated with recombinant rbST. To comply with the public demand to obtain, for example, milk and meat that can be labeled as “rBGH-free,” tests need to be developed that can discriminate food products derived from cattle treated or not treated with rbST.

### BRIEF SUMMARY

In one aspect, the disclosure provides a reliable method for determining whether a subject has been treated with exogenous somatotropin, the method comprising providing a sample from the subject; determining a level of somatotropin in the sample; comparing the determined level of somatotropin with a level of somatotropin in a reference; and determining whether the subject has been treated with exogenous somatotropin if the level of somatotropin is increased in the sample of the subject, when compared to the reference.

The biological sample preferably is a bodily fluid, preferably blood or milk.

The subject can be a fish, such as a salmon, tuna, and rainbow trout. In a preferred embodiment, the subject is a mammal, including human, a camelid such as a camel and a dromedary, a horse, and a ruminant such as a bovine, a sheep, and a goat. A preferred subject is a bovine, preferably a cow.

In a preferred method according to the disclosure, a level of bovine somatotropin is determined with the aid of an antibody or a functional part thereof directed against bovine somatotropin, preferably followed by mass spectrometry.

In one embodiment, the exogenous somatotropin is recombinant bovine somatotropin. A level of recombinant bovine somatotropin is preferably determined with the aid of an antibody or a functional part thereof directed against recombinant bovine somatotropin.

In a further preferred method according to the disclosure, the somatotropin is concentrated by affinity chromatography, preferably by repeated incubation with beads or monolithic material comprising affinity partners for somatotropin under circumstances that allow binding of somatotropin to the affinity partners.

In a preferred method according to the disclosure, somatotropin is digested, preferably with trypsin, preferably for a period of between 0.2 and 5 hours.

In a further preferred method according to the disclosure, the antibody directed against recombinant bovine somatotropin is generated by the subject.

A further preferred method according to the disclosure comprises determining a level of expression of at least one somatotropin-responsive gene product in the sample from the subject; comparing the determined level of expression of the at least one somatotropin-responsive gene product with a level of expression of the at least one somatotropin-responsive gene product in a reference; and determining whether a subject has been treated with exogenous somatotropin if the level of expression of the at least one somatotropin-responsive gene product differs from the level of expression as determined in the reference.

The at least one somatotropin-responsive gene product is preferably selected from insulin-like growth factor 1 (IGF1), anti-rbST-antibodies, osteocalcin, and/or IGFBP2.

At least one somatotropin-responsive gene product more preferably comprises insulin-like growth factor 1 (IGF1), more preferably insulin-like growth factor 1 (IGF1) in combination with endogenous anti-rbST-antibodies.

In a further preferred method according to the disclosure, the level of somatotropin is determined by Enzyme-Linked Immuno-Sorbent Assay (ELISA) or flow cytometric immunoassay (FCIA).

### BRIEF DESCRIPTION OF THE DRAWINGS

FIG. 1: Amino acid sequence of bovine growth hormone 1 (UniProtKB: P01246 (SOMA_BOVIN)).

FIG. 2: Peak areas obtained with LC-MS/MS transition m/z 913.1>m/z 774.13 after enrichment of rbST in spiked serum samples at: 0, 2, 4, 6, 8, 10, 50 and 100 ng mL-1. The observation of a plateau beyond 50 ng mL-1 indicates saturation of the binding sites of the micro-columns.

FIG. 3: Panel A: Top bar shows the results of the screenings analysis; Panel B: LC-MS/MS confirmation of biomarker screening results. Chromatograms of transition m/z 913.1>m/z 774.13 and m/z 913.1>m/z 1047.6 after enrichment of rbST in serum in (from left to right): a serum sample prior to treatment (day 0), a serum sample taken the first day of treatment (determined to contain 9.0 ng mL-1 rbST), a serum sample taken 7 days after treatment (determined to contain 1.9 ng mL-1 rbST), a serum sample taken 14 days after treatment (determined to contain <1 ng mL-1) and a serum sample spiked with 1 ng mL-1 rbST. All y-axes are scaled to the serum sample of the first day of treatment. Below the chromatogram, the compliance with the ion ratio and retention time criteria is indicated (the ion ratio and retention time interval as determined according to the 2002/657/EC are given in brackets).

FIG. 4: Calibration curves with respectively 0, 2, 4, 6, 8 and 10 ng mL-1 of rbST spiked in NaOH (black circle) to simulate enrichment elution conditions and after immuno-affinity enrichment of fortified serum samples (black square).

FIG. 5: Treatment schedule and sampling time points for animal studies I and II. Arrows indicate the treatment of the cows with rbST in slow-release formula or the slow-release formula only; bold vertical lines indicate blood sampling time points.

FIG. 6: Schematic overview of the method according to the disclosure.

FIG. 7: Standard curves of the three rbST-dependent biomarkers IGF-1, IGFBP2 and osteocalcin. Each data point is the mean of eight separate measurements in a serum-matched buffer (80 mg mL-1 BSA in PBS solution). All curves relate to 80 times diluted sera.

FIG. 8: Biomarker profiles of rbST-treated (left) and untreated (right) dairy cows. Profiles from animal study I (dotted lines) and animal study II (solid lines) are shown. Sera from adaption period (three sera from every cow), treatment period (thirteen sera per cow from animal study I and nine sera per cow from animal study II) and withdrawal period (five sera per cow from animal study I and six sera per cow from animal study II) were measured in duplicate. Biomarkers shown are concentrations of IGF-1 (Panel A), B/B0 levels of IGFBP2 (Panel B), B/Bd levels of antibodies against rbST (Panel C) and concentrations of osteocalcin (Panel D). The rbST treatment schedules for both animal studies are indicated by two black horizontal bars and decision limit per biomarker by the green horizontal line. Note that cows from animal study II received two additional rbST injections after the biweekly treatment period.

FIG. 9: Predictive power of each single candidate biomarker for indicating rbST abuse. True-positive rates were calculated for all samples from rbST-treated cows in their treatment and withdrawal periods of study I and II. False-positive rates were calculated for all samples from untreated cows from the two animal studies (adaptation period samples from all cows and all the samples from untreated cows). Samples were considered positive if their biomarker value exceeded the respective decision limit. The treatment schedules of the two controlled animal studies are indicated by black horizontal bars on top of the graph. The targeted 95% true-positive (<5% false-compliant) rate according to 2002/657/EC is indicated by the dotted horizontal line.

FIG. 10: Number of biomarkers reacting above the respective decision limit. Results shown per cow (in animal studies I and II) and day of the controlled animal studies. Each row represents one individual cow. Vertical dotted lines indicate the treatment time points in both animal studies.

FIG. 11: Predictive power (shown as true-positive and false-positive rates) of the additive biomarker analysis. True-positive rates were calculated for all samples from rbST-treated cows in their treatment and withdrawal periods of study I and II. False-positive rates were calculated for all samples from untreated cows from the two animal studies (adaptation period samples from all cows and all the samples from untreated cows). Samples were considered positive if one of the candidate biomarkers exceeded its respective decision limit. The treatment schedules of the two animal studies are indicated by black horizontal bars on top of the graph. The targeted 95% true-positive (<5% false-compliant) rate according to 2002/657/EC is indicated by the dotted horizontal line.

FIG. 12: True-positive rates following statistical multiple biomarker analysis. True-positive rates, obtained with the prediction models based on the eleven different biomarker combinations, were calculated for rbST-treated cows from animal study I in their treatment and withdrawal period. The treatment schedules of animal study I is indicated by black horizontal bars on top of the graphs. The targeted 95% true-positive rate according to 2002/657/EC is indicated by the dotted horizontal lines.

### DETAILED DESCRIPTION

### 1. Abbreviations

The term “subject,” as is used herein, refers to a fish, such as a salmon, tuna, rainbow trout, a mammal, including human, a camelid such as a camel and a dromedary, a horse, and a ruminant such as a bovine, a sheep, and a goat. A preferred subject is of the biological subfamily Bovinae, preferably Bos taurus.

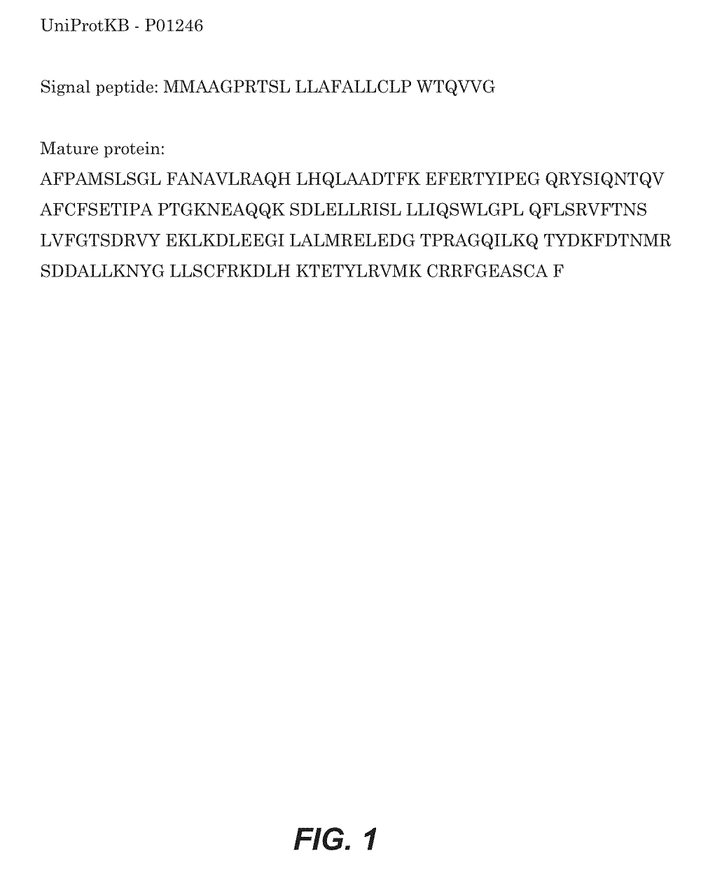

The term “somatotropin” or “growth hormone,” as is used herein, refers to a peptide hormone that stimulates growth, cell reproduction, and cell regeneration in humans and other animals. Human growth hormone is a 191-amino acid, single-chain polypeptide that is synthesized in the pituitary gland. Bovine growth hormone also is a 191-amino acid, single-chain polypeptide. An amino acid sequence of bovine growth hormone 1 (UniProtKB: P01246 (SOMA_BOVIN)) is provided in FIG. 1.

The term “exogenous somatotropin,” as is used herein, refers to somatotropin that is administered to a subject. The exogenous somatotropin preferably is recombinant somatotropin, most preferably recombinant bovine somatotropin (rbST).

The term “treating” or “treatment,” as is used herein, refers to the administration of exogenous somatotropin to a subject.

The term “sample,” as is used herein, refers to a sample from a subject that is, or comprises, a biological fluid that contains somatotropin, including exogenous somatotropin, and/or that contains markers indicating that the subject has been treated with exogenous somatotropin. The biological fluid preferably is milk, blood, synovial fluid, urine, spinal fluid, bronchiolar lavage fluid, lymph, extracts of tissues such as muscle, or extracts of bones or cartilage.

The term “a level of somatotropin,” as is used herein, refers to the amount of endogenous and exogenous peptide hormone that is present in a sample. The level preferably is expressed in ng/ml. A calibration curve including known amounts of exogenous somatotropin preferably is included in an analysis.

The term “reference,” as is used herein, refers to a biological fluid that contains somatotropin, and/or that contains markers indicating that the subject has been treated with exogenous somatotropin, or not. The biological fluid preferably is milk, blood, synovial fluid, urine, spinal fluid, bronchiolar lavage fluid, lymph, extracts of tissues such as muscle, or extracts of bones or cartilage.

The term “antibody” as used herein, refers to an immunoglobulin protein comprising at least a heavy chain variable region (VH), paired with a light chain variable region (VL), that is specific for a target epitope.

A “functional part of an antibody” is defined herein as a part that has at least one shared property as the antibody in kind, not necessarily in amount. Non-limiting examples of a functional part of an antibody are a single domain antibody, a single chain antibody, a nanobody, a unibody, a single chain variable fragment (scFv), an Fd fragment, a Fab fragment and a F(ab′)2 fragment.

The term “specifically recognizes and binds” refers to the interaction between an antibody, or functional part or functional equivalent thereof, and its epitope on a protein or peptide. This means that the antibody, or functional part or functional equivalent thereof, preferentially binds to the epitope over other antigens or amino acid sequences. Thus, although the antibody, functional part or equivalent may non-specifically bind to other antigens or amino acid sequences, the binding affinity of the antibody or functional part or functional equivalent for its epitope is significantly higher than the non-specific binding affinity of the antibody or functional part or functional equivalent for other antigens or amino acid sequences.

The term “amplification” or “amplify,” as is used herein, refers to the in vitro amplification of a specific nucleic acid sequence, such as to test for presence of rbST or of at least one somatotropin-responsive gene in a sample. In vitro amplification methods include amplification of a target nucleic acid sequence using, for example, ligase chain reaction (LCR), isothermal ribonucleic acid amplification such as nucleic acid sequence-based amplification (NASBA) and cleavage-based signal amplification of RNA, transcription mediated amplification, strand displacement amplification and, preferably, polymerase chain reaction (PCR). An amplification is preferably specific, meaning that only a region between two hybridization nucleic acid sequences is amplified. An amplification reaction preferably is performed in a reaction chamber such as a vial or a well of a microtiter plate, or a fluidic chamber of a cartridge system.

The term “PCR reaction,” as is used herein, refers to an amplification reaction that is characterized by repeated cycles of denaturation of target nucleic acid template, annealing of primers, and extension (synthesis) of new nucleic acid strand. The specificity of a PCR reaction is substantially determined by the % identity of the primers to the target nucleic acid template and the annealing temperature.

The term “quantitative PCR,” as is used herein, refers to a PCR amplification reaction to which a labeled probe or a dye is added to generate a signal. The intensity of the signal is a measure for the amount of product that is generated. Detection of the signal in real-time allows quantification of the amount of starting material.

### 2. Exogenous Growth Hormone

Growth hormone, also termed somatotropin, is a hormone that is produced by the anterior pituitary gland. The hormone is known to stimulate mammary gland growth and to regulate milk production. Administration of exogenous somatotropin, such as recombinant bovine somatotropin (rbST), enhances milk production in dairy cattle. Use of rbST is approved in several countries including the United States, but is prohibited in, for example, the European Union.

The identification of a subject that has been treated with rbST is a challenge due to the similarity of rbST with endogenous hormone (bST). Further challenges are provided by the low concentration of rbST in milk and blood after administration to the animal, and the short half-life of rbST in animal products, especially after heat treatment. For example, endogenous growth hormone or somatotropin has been detected in milk at concentrations lower than 1 ng mL-1 (Torkelson, 1987, Proc. Am. Dairy. Sci. Assoc. 70:146), and it is not considered likely that concentrations of exogenous growth hormone will be much higher in milk.

Natural bovine growth hormone is synthesized as a pre-hormone of 217 amino acids, of which a 26 amino acid signal peptide is cleaved of when the hormone is released from the pituitary gland. The N-terminus of the mature 191 amino acid hormone comprises the amino acids residues N-alanine-phenylalanine-proline (N-Ala-Phe-Pro or N-AFP). In some recombinant variants of the hormone the N-terminal part is changed or enlarged, e.g., the alanine is replaced by one or more amino acids.

In one form of recombinant bovine somatotropin, alanine is replaced by methionine, resulting in N-methionine-phenylalanine-proline (N-Met-Phe-Pro or N-MFP) as the N-terminal amino acid residues of recombinant bovine somatotropin. In a second form of recombinant bovine somatotropin, alanine is replaced by methionine-aspartic acid-glutamine, resulting in N-methionine-aspartic acid-glutamine-phenylalanine-proline (N-Met-Asp-Gln-Phe-Pro or N-MDQFP) as the N-terminal amino acid residues of recombinant bovine somatotropin. In a third form of recombinant bovine somatotropin, alanine is replaced by methionine-phenylalanine-proline-leucine-aspartic acid-aspartic acid-aspartic acid-aspartic acid-aspartic acid-lysine, resulting in N-methionine-phenylalanine-proline-leucine-aspartic acid-aspartic acid-aspartic acid-aspartic acid-aspartic acid-lysine-phenylalanine-proline (N-Met-Phe-Pro-Leu-Asp-Asp-Asp-Asp-Asp-Lys-Phe-Pro or N-MFPLDDDDDKFP (SEQ ID NO:1)) as the N-terminal amino acid residues of recombinant bovine somatotropin. In a fourth form of recombinant bovine somatotropin, alanine is replaced by methionine-phenylalanine-proline-leucine-aspartic acid-aspartic acid-aspartic acid-aspartic acid-lysine, resulting in N-methionine-phenylalanine-proline-leucine-aspartic acid-aspartic acid-aspartic acid-aspartic acid-lysine-phenylalanine-proline (N-Met-Phe-Pro-Leu-Asp-Asp-Asp-Asp-Lys-Phe-Pro or N-MFPLDDDDKFP (SEQ ID NO:1)) as the N-terminal amino acid residues of recombinant bovine somatotropin (Secchi and Borromeo, 1997, J. Chrom. B. 688:161-177). In still another form of the recombinant bovine somatotropin, the N-terminus of the recombinant form is identical to the natural mature bovine somatotropin.

The presence of an amino-terminal methionylated analogue (N-Met-x-x-x) in recombinant somatotropin provides an opportunity for a reliable detection method to differentiate between samples from a subject that was treated with exogenous, recombinant somatotropin and a sample from a subject that was not treated with exogenous, recombinant somatotropin. The reliable detection method preferably is based on a difference in mass between a fragment comprising the N-terminal alanine in natural bST, and a fragment comprising the N-terminal methionine in exogenous, recombinant bST. In addition to chemical production methods, recombinant bovine somatotropins are produced in micro-organisms, e.g., bacteria such as Escherichia coli and yeasts such as Saccharomyces cerevisiae, as is indicated in U.S. Pat. No. 5,366,876. The production of recombinant bovine somatotropin in bacteria and/or lower eukaryotes results in differences in post-translational modification of recombinant bovine somatotropin, when compared to endogenous natural mature bovine somatotropin. This difference offers a further opportunity to discriminate between exogenous, recombinant bovine somatotropin and endogenous natural mature bovine somatotropin.

### 3. Detection of Exogenous rbST Growth Hormone

This disclosure is directed to a method for determining whether a subject has been treated with exogenous somatotropin, the method comprising the following steps: providing a sample from the subject; determining, directly and/or indirectly, a level of somatotropin in the sample; comparing the determined level of somatotropin with a level of somatotropin in a reference; and determining whether the subject has been treated with exogenous somatotropin if the level of somatotropin is increased in the sample of the subject, when compared to the reference.

The sample preferably is pretreated to remove contaminants and to increase the concentration of the rbST. This will result in a lower detection limit and will improve reliability of the assay.

The reference preferably is a sample from a non-treated animal.

If the determined level of somatotropin is found to be increased, when compared to a reference, the animal is defined as suspected of being treated with exogenous somatotropin. An additional confirmation step which is in compliance with definition as stated in Commission Decision 2002/657 is preferably performed to determine unambiguously the presence of rbST.

Preferably, the sample is a bodily fluid, preferably blood or milk, but could also comprise urine, saliva or other bodily fluids. The sample is collected from the subject and is treated to maintain the integrity, natural state of proteins and/or peptides. Methods for generating a proteinaceous sample are known to the skilled person.

Preferred methods, for example, of a sample comprising milk, include the removal of cells by, for example, centrifugation. A milk sample preferably is centrifuged at low speed, such as between 2000×g and 5000×g, preferably at about 3000×g. Centrifugation preferably is performed at reduced temperature, preferably between 0° C. and 10° C., such as 4° C. Samples may also be pre-treated by ultracentrifugation.

A blood sample preferably is or comprises serum, which is prepared, for example, by coagulation of platelets, for example, at room temperature, followed by centrifugation at low speed, such as between 2000×g and 5000×g, preferably at about 3000×g. Centrifugation preferably is performed at a room temperature, preferably between 20° C. and 25° C.

A tissue sample preferably is disrupted for example, by homogenization, for example, by application of pressure, ultrasound or by mechanical homogenization, as is known to the skilled person.

The proteinaceous sample may be fractionated used standard techniques such as chromatography methods including ion exchange chromatography and/or size-exclusion chromatography, as is known to the skilled person.

3.1 Direct rbST Detection

For direct determination of a level of somatotropin in the sample, somatotropin preferably is concentrated by affinity chromatography, for example, by employing affinity partners such as antibodies or functional parts thereof that bind specifically to exogenous somatotropin or to both endogenous and exogenous somatotropins. The concentration step preferably removes proteins and/or peptides that interfere with the subsequent detection of somatotropin.

Affinity partners such as antibodies or functional parts thereof that bind specifically to somatotropin are preferably not directed against the N-terminus of the protein, as this region differs between the various available recombinant somatotropins. The affinity partners, such as antibodies or functional parts thereof, that bind specifically to somatotropin are preferably directed against the C-terminal part of somatotropin, preferably against epitopes within the C-terminal stretch of approximately 180 amino acids.

Affinity partners such as antibodies or a functional part thereof that bind specifically to both endogenous and exogenous somatotropin preferably do not bind to the N-terminus of somatotropin, but specifically recognize and bind to the central or C-terminal part of the protein only.

Preferred antibodies or functional parts thereof include, but are not limited to, anti-growth hormone antibody (ab31496; ABCAM®) and OHI antibody (AP02012SU-S; Jomar Life Research).

As an alternative, a mixture of antibodies, preferably monoclonal antibodies, or functional parts thereof, may be employed. Preferred affinity partners are polyclonal antibodies, such as rabbit polyclonal antibodies obtained after injection of a rabbit with recombinant somatotropin, as is known to the skilled person. An example of a polyclonal antiserum is described in Heutmekers et al., 2007, Anal. Chim. Acta. 586:239-245.

Several methods may be employed to concentrate somatotropin by affinity chromatography or immunoprecipitation using, for example, the affinity partners, such as antibodies or functional parts thereof. It is preferred that the affinity partners such as antibodies or functional parts thereof are coupled to a carrier material such as beads, preferably magnetic beads, or to monolithic material, preferably monolithic material that is embedded in columns, preferably in micro-columns.

The affinity partners may be coupled directly to the beads or monolithic material, or indirectly, for example, by coupling of a second antibody that specifically recognizes the somatotropin-specific antibody. Preferably, the antibodies are indirectly coupled to the beads or monolithic material by coupling of protein A, protein G, or a mixture of protein A and G to the beads or to the monolithic material. The antibodies, preferably polyclonal antibodies, are preferably coupled to protein A-coupled beads or protein A-coupled monolithic material.

The proteinaceous sample comprising somatotropin is incubated with the beads or monolithic material under circumstances that allow binding of somatotropin to the affinity partners on the beads or monolithic material. It is preferred that the proteinaceous sample is repeatedly incubated with the beads or monolithic material under circumstances that allow binding of somatotropin to the affinity partners, preferably at least 100 times, more preferred at least 1000 times, such as 1500 times, 2000 times, 5000 times, most preferred 10,000 times. Following the repetitive incubation steps, the beads or monolithic material are washed, for example, with phosphate-buffered saline or with a 20 mM phosphate buffer at pH 7. The washing step preferably is repeated, preferably two to twenty times, more preferably about ten times.

Following concentration of somatotropin by affinity chromatography, the concentrated protein is released from the affinity partners. Release of somatotropin may be accomplished by any method known in the art, including the application of a high pH buffer, a low pH buffer and/or a high salt buffer. A preferred elution buffer comprises 200 mM NaOH. The elution step preferably is repeated, preferably 50-1000 times, more preferably 100-500 times, most preferably about 150 times. After collection of the eluate, a buffered solution such as a 50 mM Tris pH 7.9 preferably is added before further use, rendering the pH>10. Eluates can be stored at −20° C. until further use.

After release, somatotropin is digested, for example, with trypsin, to generate a tryptic digest that includes a fragment comprising the N-terminal amino acid (alanine) of natural bST and, if present, a fragment comprising the N-terminal amino acid of exogenous, recombinant bST. Digestion may be performed by application of conditions known to the skilled person. The pH of the obtained solution may be adjusted to 8-8.5, for example, with 1 M HCl. Sulphur bridges are preferably reduced, for example, by addition of dl-dithiothreitol (DTT). Iodoacetamide is preferably added for methylation of cysteine residues. Amounts used are optimized to obtain the highest amount of N-terminal rbST peptide to obtain at the end the unique sensitivity needed to determine rbST.

The digestion time may be between 0.2 and 5 hours. Preferably the digestion time is about 1 hour to obtain sufficient digested N-terminal amino acid fragments. Digestion may be stopped, for example, by the addition of formic acid. The digest may be concentrated, for example, on an Agilent Bond Elut Plexa SPE column.

Presence or absence of a fragment comprising the N-terminal alanine in natural bST and of a fragment comprising the N-terminal methionine in exogenous, recombinant bST is preferably determined by determining the relative molecular mass of peptides in the sample by a first stage mass spectrometry, after which the peptides are fragmented and fragment ions analyzed by second stage of the tandem mass spectrometry.

A preferred method for determining a level of somatotropin, preferably bovine somatotropin, is based on mass spectrometry (MS). MS preferably is used to detect a level of somatotropin in a sample of a subject, preferably by detecting exogenous bST, or more preferably, both endogenous bST and exogenous bST.

A preferred method for determining a level of somatotropin comprises ultra-high performance liquid chromatography (UHPLC) coupled to tandem mass spectrometry (LC-MS/MS) in positive electrospray ionization mode, allowing the unambiguous identification and quantification of somatotropin, including rbST in milk, blood (serum) and/or a tissue sample. The LC-MS/MS analysis may be performed, for example, by using a high end UHPLC chromatographic system coupled to a triple-quadrupole mass-spectrometer

3.2 Indirect Detection

3.2.1 Antibodies Against Recombinant Somatotropin

It has been documented that a subject that has been treated with recombinant somatotropin, such as rbST, develops antibodies against the recombinant somatotropin (Smits et al., 2012, Drug Testing and Analysis 4:362-367). The presence of these antibodies can be used to indirectly determine recombinant somatotropin treatment of the subject.

To detect an antibody directed against recombinant bovine somatotropin that is generated by the subject, preferably an immunoassay is performed. A preferred immunoassay is an enzyme-linked immunosorbent assay (ELISA). For this, recombinant somatotropin, or parts thereof, may be absorbed or coated onto a solid phase, for example, a microtiter plate, and immobilized. Following incubation of the solid phase with a serum or milk sample from the subject, the presence of antibodies from the subject that have bound to the immobilized recombinant somatotropin, the so called primary antibodies, can be detected by addition of a secondary antibody that specifically recognize and bind to the primary antibody and which is coupled to a label or an enzyme. The label can be a radioactive tag, a chromogenic tag, or a chemiluminescent tag, for example, a fluorescent tag. The enzyme, for example, horseradish peroxidase, preferably is detected by the introduction of an enzyme substrate, which is converted by the enzyme into a detectable product.

A preferred assay to detect an antibody directed against recombinant bovine somatotropin that is generated the subject is a microsphere-based flow cytometric immunoassay (FCIA).

Non-specific binding of proteins, for example, in a sample comprising serum or milk, to recombinant somatotropin results in a high rate of false positive results. This non-specific binding preferably is reduced or eliminated, for example, by pretreatment with a glycine solution, for example, by diluting with glycine or with glycine and bovine serum albumin under constant mixing, preferably vortexing.

3.2.2 Marker Proteins

A preferred method for determining whether a subject has been treated with exogenous somatotropin comprises, in addition to direct and/or indirect detection of recombinant somatotropin, comprises the determination of a level of expression of at least one somatotropin-responsive gene product in a sample from the subject.

The somatotropin-responsive gene products found to be changed in expression upon treatment with rbST are insulin-like growth factor 1 (IGF1; increased in milk from rbST-treated cows), myostatin (reduced in muscle from rbST-treated cows; Liu et al., 2003, J. Clin. End. Metab. 88:5490-5496), fibroblast growth factor 21 (FGF21; induced by growth hormone; Chen et al., 2011, J. Biol. Chem. 286:34559-34566) and/or markers described in Ludwig et al., 2012, PLoS ONE 7(12):e52917, including acid labile subunit (ALS), apolipoprotein A-1 (APOA1), C-terminal cross-linked telopeptide of collagen I (ICTP), C-terminal propeptide of procollagen I (PICP), hemoglobin a-chain (HbA1), IGF binding protein 2 (IGFBP2), IGF binding protein 3 (IGFBP3), inter-alpha-trypsin inhibitor heavy chain H4 (ITIH4), leucine-rich a-2-glycoprotein (LRG), anti-rbST antibodies, N-terminal propeptide of procollagen I (PINP), N-terminal propeptide of procollagen III (PIIINP), osteocalcin, transthyretin (TTR) and alpha-1 antitrypsin (AAT).

The somatotropin-responsive gene products found to be changed in expression upon treatment with rbST preferably include insulin-like growth factor 1 (IGF1) and anti-rbST-antibodies, anti-rbST-antibodies and osteocalcin, and/or IGF-1, preferably, anti-rbST-antibodies and osteocalcin, more preferably IGF-1, IGFBP2, anti-rbST-antibodies and osteocalcin.

Methods for determining a level of expression of IGF1, myostatin, FGF21, anti-rbST-antibodies. ALS, APOA1, ICTP, PICP, IGFBP2, IGFBP3, ITIH4, LRG, PINP, PIIINP, osteocalcin, TTR and/or AAT are known to the skilled person, including ELISA, FCIA, FRET, dipstick, SPR, quartz crystal, microbalance, and any other acoustic, photonic, plasmonic, electrochemical version thereof, either in direct mode or resonance mode using commercially available antibodies or functional parts thereof. Other methods for determining the level of expression are available such as mass-spectrometric detection.

A preferred method for determining a level of expression of IGF1, myostatin, FGF21, anti-rbST-antibodies. ALS, APOA1, ICTP, PICP, IGFBP2, IGFBP3, ITIH4, LRG, PINP, PIIINP, osteocalcin, TTR and/or AAT comprises peptide profiling, for example, using PEPTRIX™ (available from sourceforge.net/projects/peptrix). Peptide profiles of tissue samples, for example, a milk sample or a meat sample, can be generated using, for example, reversed-phase, magnetic particle-assisted sample processing coupled to a matrix-assisted laser desorption/ionization-time of flight MS readout. As an alternative, an ORBITRAP®-based mass spectrometer, for example, equipped with an electrospray ionization source, can be used to obtain peptide profiles from a tissue sample of a bovine animal to determine whether or not the animal has been treated with exogenous somatotropin. The determined peptide profile is compared with a profile of a reference.

As an alternative, a level of expression of IGF1, myostatin, FGF21, ALS, APOA1, ICTP, PICP, IGFBP2, IGFBP3, ITIH4, LRG, PINP, PIIINP, osteocalcin, anti-rbST-antibodies, TTR and/or AAT can be determined by quantitative amplification such as qPCR. Primers for amplification of messenger RNA molecules, or of copy-DNA complements thereof, of IGF1, myostatin, FGF21, ALS, APOA1, ICTP, PICP, IGFBP2, IGFBP3, ITIH4, LRG, PINP, PIIINP, osteocalcin, TTR and/or AAT are available or can be determined by the skilled person based on the published nucleotide sequences, preferably the bovine nucleotide sequences.

Methods for the design of primers and probes are known in the art. For example, Premier Biosoft (Palo Alto, Calif., USA) offers ALLELEID® and BEACON DESIGNER™ to design probes for real-time PCR assays that are free of dimers, repeats and runs and ensure signal fidelity. In addition, Primer3 (available at http://primer3.sourceforge.net) and Integrated DNA Technologies, Inc. (www.idtdna.com/Scitools/Applications/Primerquest) provide online tools for the design of primers and probes for real-time PCR assays. Hence, the skilled person is able to design primers and probes for real-time PCR analyses of at least one somatotropin-responsive gene.

For this ribonucleic acid (RNA) may be isolated from a sample using, for example, the Ambion Ribopure™ kit or the QIAGEN® RNEASY® kit. The RNA can be freshly prepared from the sample, or it can be prepared from samples that were stored at −70° C. until processing.

Alternatively, samples can be stored under conditions that preserve the quality of the protein or RNA. Examples of these preservative conditions are fixation using, e.g., formalin and paraffin embedding, RNase inhibitors such as RNASIN® (Pharmingen) or RNASECURE™ (Ambion), aqueous solutions such as RNALATER® (Assuragen; U.S. Pat. No. 6,204,375) and non-aqueous solutions such as Universal Molecular Fixative (Sakura Finetek USA Inc.; U.S. Pat. No. 7,138,226).

RNA is preferably converted into complementary DNA (cDNA) prior to amplification, using a RNA-dependent DNA polymerase or reverse transcriptase. Methods for the conversion of RNA into cDNA are known in the art and include recombinant M-MuLV reverse transcriptase or AMV reverse transcriptase. Suitable commercially available systems for cDNA synthesis include commercially available systems for DNA isolation are used, such as the QSCRIPT® cDNA synthesis kit (Quanta Biosciences, Gaithersburg, Md.) and the Maxima H Minus First Strand cDNA Synthesis Kit (Thermo Fisher Scientific Inc., Waltham, Mass.). It is preferred that random primers, for example, hexamers or nonamers, or gene-specific primers are used for cDNA synthesis.

Different amplification methods, known to a skilled artisan, can be employed for amplification, including but not limited to Polymerase Chain Reaction (PCR), rolling circle amplification, nucleic acid sequence-based amplification, transcription mediated amplification, and linear RNA amplification. A preferred amplification method is PCR, including end-point PCR and, preferably, quantitative (real time) PCR.

3.3 Reference Sample

A level of expression of somatotropin and/or of at least one somatotropin-responsive gene product selected from IGF1, myostatin, FGF21, ALS, APOA1, ICTP, PICP, IGFBP2, IGFBP3, ITIH4, LRG, PINP, PIIINP, osteocalcin, TTR and/or AAT is preferably compared with a level of somatotropin in a reference. The reference preferably comprises a tissue sample or a biological fluid from a subject that is known to be treated, or known not to be treated, with exogenous somatotropin, or a tissue sample or biological fluid from a group of subjects that are known to be treated, or known not to be treated, with exogenous somatotropin. The levels of expression preferably are stored on a computer, or on computer-readable media, to be used in comparisons to the level of expression level data from the sample of the subject.

The level of expression of somatotropin and/or of a somatotropin-responsive gene product may differ between subspecies or even between breeds. Preferably, the reference, therefore, is of the same subspecies or breed as the subject of which it is to be determined whether it has been treated with exogenous somatotropin, or not. For example, in the case that the subject is a zebu, the reference preferably also is a zebu. Similarly, if the subject is a Frisian Holstein, the reference preferably also is a Frisian Holstein. If the subject is a Dutch belted cow (Lakenvelder), the reference preferably also is a Lakenvelder.

Based on the comparison with the level of expression of the at least one somatotropin-responsive gene product in the reference, it can be determined whether a subject has been treated with exogenous somatotropin if the level of expression of at least one somatotropin-responsive gene product differs from the level of expression as determined in the reference.

When a level of bovine somatotropin is determined with the aid of an antibody or a functional part thereof directed against bovine somatotropin, the internal control preferably is a known amount of a peptide that is specifically recognized and bound by the antibody, but which differs from the endogenous and exogenous somatotropins.

Similarly, if antibodies are used for determining a level of at least one somatotropin-responsive gene product, an internal control preferably is a known amount of a peptide that is specifically recognized and bound by the antibody, but which differs from the somatotropin-responsive gene product. The amount of the peptide can be determined using any method known to the skilled person, including ELISA, FCIA, FRET, dipstick, SPR, quartz crystal, microbalance, and any other acoustic, photonic, plasmonic, electrochemical version thereof, either in direct mode or resonance mode using commercially available antibodies or functional parts thereof. Other methods for determining the level of expression are available such as mass-spectrometric detection as used, for example, in peptide profiling.

In the case that quantitative amplification such as qPCR is used to determine a level of expression of IGF1, myostatin, FGF21, ALS, APOA1, ICTP, PICP, IGFBP2, IGFBP3, ITIH4, LRG, PINP, PIIINP, osteocalcin, anti-rbST-antibodies, TTR and/or AAT, the internal control preferably is a nucleotide molecule that harbors nucleotide sequences that are complementary to the primers that are used for amplification. For example, in the case that primers A and B are used for determining a level of expression of IGF1, a nucleotide molecule harboring sequences that are complementary to the sequences of A and B may be added as an internal control to the sample. If a probe C is used to detect an amplified fragment of an IGF1 product, the complementary nucleotide sequences of probe C are preferably not present in the internal control. Preferably, the internal control harbors sequences that are complementary to probe D, which complementary sequences are not present in an IGF1 product. Hence, in the example, an IGF1 product harbors sequences complementary to ACB, or to BCA, while the internal control harbors sequences complementary to ADB, or to BDA.

The skilled person will understand that the complementary sequences to probe D preferably are not present in any one of myostatin, FGF21, ALS, APOA1, ICTP, PICP, IGFBP2, IGFBP3, ITIH4, LRG, PINP, PIIINP gene products. In that case, probe D can be used for detection of the internal control in all quantitative amplification reactions.

3.4 Kit

The disclosure further provides a kit for determining whether a subject has been treated with exogenous somatotropin, the kit comprising a device for collecting a test sample from a patient; and reagents for directly or indirectly determining a level of exogenous somatotropin in the sample.

In one embodiment, the reagents for determining a level of exogenous somatotropin preferably are reagents for real-time PCR including primers and include, for example, first and second primers, buffer, dNTPs, DNA polymerase, preferably in lyophilized form. These reagents preferably enable determination of a level of expression of at least one somatotropin-responsive gene product.

In one embodiment, the reagents for determining a level of exogenous somatotropin preferably are reagents for an immunochemical assay.

It is preferred that the reagents for determining a level of exogenous somatotropin include a receptacle that is coated with recombinant bovine somatotropin, or monolithic material or microbeads that are coated with recombinant bovine somatotropin, allowing detection of antibodies directed against recombinant bovine somatotropin that are generated by the subject.

It is further preferred that the reagents for determining a level exogenous somatotropin include a receptacle that is coated with antibodies against at least one somatotropin-responsive gene product, or monolithic material or microbeads that are coated with antibodies against at least one somatotropin-responsive gene product, allowing detection of a level of expression of at least one somatotropin-responsive gene product.

The monolithic material or microbeads are preferably coated with antibodies against at least two somatotropin-responsive gene products, more preferably at least three somatotropin-responsive gene products, more preferably at least four somatotropin-responsive gene products, most preferably at least five somatotropin-responsive gene products, such as six somatotropin-responsive gene products, seven somatotropin-responsive gene products and ten somatotropin-responsive gene products.

The monolithic material or microbeads coated with multiple antibodies enable simultaneous detection of multiple somatotropin-responsive gene products. The simultaneous analysis is cost effective and amenability to high-throughput/automation.

The disclosure further provides a use of a kit according to the disclosure for determining whether a subject has been treated with exogenous somatotropin

3.5 Timing

Due to the extreme sensitive methodology of the disclosure, it is possible to detect rbST from 4 hours to 14 days after administration, this equals the time between two sequential administrations of exogenous somatotropin as advised by the manufactures of rbST. Detection of antibodies that are directed against recombinant bovine somatotropin, and which are generated by the subject, is likely possible at least from 6 hours to 21 days after administration of exogenous somatotropin. Detection of at least one somatotropin-responsive gene product will similarly be possible from 6 hours to 10 days after administration of exogenous somatotropin.

3.6 General

For the purpose of clarity and a concise description, features are described herein as part of the same or separate aspects and preferred embodiments thereof, however, it will be appreciated that the scope of the disclosure may include embodiments having combinations of all or some of the features described.

The disclosure will now be illustrated by the following examples, which are provided by way of illustration and not of limitation and it will be understood that many variations in the methods described and the amounts indicated can be made without departing from the spirit of the disclosure and the scope of the appended claims.

### EXAMPLES

### Example 1. Direct Detection

### Materials and Methods

### Materials

Monsanto rbST standard was obtained from the National Hormone & Peptide Program (NHPP) of Dr. Parlow (Torrance, Calif.). Elanco rbST was obtained from Elanco (Indianapolis, Ind., USA). Lactotropin 500 mg single-dose syringes were purchased from Centro de Tecnologia (Rio de Janeiro, Brazil).

Pierce BCA protein assay, the FINNPIPETTE® Novus i Multichannel Electronic and monolithic micro-columns (MSIA disposable automation research tips (D.A.R.T.), containing approximately 10 mg packed bed Protein A or Protein A/G) were all purchased from Thermo Fisher Scientific (Rockford, Ill.). Ammonium sulphate, hydrochloric acid, potassium phosphate, sodium hydroxide, sodium phosphate and the ultrasonic cleaner were purchased from VWR International (Amsterdam, The Netherlands). Trypsin, tris(hydroxymethyl)aminomethane, iodoacetamide (IAA), dimethyl sulfoxide (DMSO) and dl-dithiothreitol were purchased from Sigma-Aldrich Chemie (Zwijndrecht, The Netherlands). Methanol and acetonitrile were purchased from Biosolve (Valkenswaard, The Netherlands). Formic acid was purchased from Actu-All chemicals (Oss, The Netherlands). Protein Lobind Tubes (1.5 mL, 2.0 mL) and a table centrifuge model 5810R were obtained from Eppendorf (Hamburg, Germany). The Jouan GR 20-22 ultracentrifuge was obtained from Jouan (Saint-Herblain, France). The Snijder test tube rotator was purchased from Omnilabo International (Breda, The Netherlands).

An isotopic-labelled bST peptide AFPAMSLSGLFANAVLR (SEQ ID NO:) and a synthetic analogue of the rbST peptide MFPAMSLSGLFANAVLR (SEQ ID NO:) were obtained from Bachem (Bubendorf, Switserland). The LC-column: Kinetex 50×2.10 mm I.D. 1.3 μm C18 (100 Å) was purchased from Phenomenex (Utrecht, the Netherlands). Bond Elut Plexa 30 mg solid-phase extraction columns were purchased from Agilent Technologies (Amstelveen, The Netherlands). A Zymark TurboVap was purchased from Biotage (Upsala, Sweden).

### Serum Samples

Serum samples from two controlled animal treatment studies were used. In the first animal treatment study, serum samples were obtained from one three-year-old dairy cow (a) treated twice with subcutaneous injections of 500 mg Lactotropin. This treatment was part of a sequential Lactotropin-steroid treatment schedule existing of three compounds in total. Of each compound, two subcutaneous injections were administered with a one-week interval. After each treatment, an adaptation period of two weeks was taken into account. Blood samples were collected daily during the week after each treatment. The second animal treatment study was according to commonly used rbST treatment conditions as recommended by the manufacturer: an adaptation period of two weeks was taken into account, and then the cow was treated every second week with 500 mg rbST, according to manufacturers' guidelines. Serum samples were obtained from one three-year-old dairy cow (b).

After blood collection, the blood sample was placed at room temperature for four hours to coagulate. After coagulation, the samples were centrifuged for 10 minutes at 3000×g, and serum was collected and stored at −20° C. until further use. The experimental procedure was authorized by the ethical committee of ID-DLO in Lelystad, the Netherlands.

### Preparation of Polyclonal Antiserum

The preparation of polyclonal antiserum against Elanco rbST was described before by Heutmekers et al., 2007, Anal. Chim. Acta. 586:239-245). Briefly, a New Zealand White rabbit (no. 58) was immunized with Elanco rbST at the Centre for Small Laboratory Animals in Wageningen, the Netherlands. Blood was obtained at various moments during the entire treatment period and serum was collected. The sera collected over the total treatment were pooled and stored at −80° C. for further use.

To concentrate the antibodies from the antiserum and to remove abundant proteins, first, the combined rabbit antiserum no. 58 was diluted three times with PBS (154 mM NaCl, 5.39 mM Na₂HPO₄, 1.29 mM KH₂PO₄, pH 7.4). Then, slowly, under constant stirring, an equal amount of saturated ammonium sulfate was added. Next, this solution was left at room temperature for 30 minutes without stirring. Subsequently, the solution was ultra-centrifuged for 10 minutes at 10,000×g, the supernatant was discarded and the pellet was re-suspended in PBS to restore the starting serum volume. Finally, the re-suspended pellet was dialyzed against PBS for 24 hours. The protein concentration was 6.6 mg mL-1 determined by the BCA protein assay according to the manufacturer's protocol.

### Coupling of Antibodies

Polyclonal anti-rbST ammonium-sulfate purified antibodies were immobilized to monolithic micro-columns loaded with, respectively, protein A or protein A/O by affinity binding. The pipet tips containing the protein A and protein A/G monolithic micro-columns were placed on a FINNPIPETTE® Novus i multichannel, which is an automated device having a repetitive cycling function. First, the monolithic micro-columns were washed ten times with 150 μL 20 mM phosphate buffer pH 7. Adherent solution was removed from the column by air pressure. Next, 75 μL 0.1 mg mL-1 of the ammonium-sulfate-precipitated polyclonal anti-rbST antibodies, resuspended in 20 mM phosphate buffer pH 7, was transferred over the column 1000 times. After the last cycle, adherent solution was removed from the column by air pressure. To remove unbound antibodies, the monolithic micro-columns were washed twice by 10 cycles of 150 μL 20 mM phosphate buffer pH 7. It took 50 minutes to obtain these freshly prepared monolith micro-columns immobilized with ammonium-sulfate-precipitated polyclonal anti-rbST antibodies, which were directly used for rbST enrichment.

### Enrichment of Somatotropin

For immuno-enrichment, 1 mL serum sample or spiked serum was diluted with 1 mL 20 mM phosphate buffer pH 7. The sample was transferred 2000 times (300 μL per cycle) through the anti-rbST-immobilized monolithic micro-column. The adherent sample was removed from the column by air pressure. To remove the remaining unbound sample, the monolithic micro-columns were washed ten times with 150-μL portions of 20-mM phosphate buffer. The captured rbST was then eluted from the monolith micro-column by 50 μL 200 mM NaOH (20 μL per cycle, 150 times). The eluate was collected, and 50 μL 50 mM Tris pH 7.9 was added before further use (final pH>10). Eluates were stored at −20° C. until tryptic digestion. The enrichment procedure took 4.5 hours.

### Digestion and Cleanup of the Immuno-Affinity-Purified Extract

For digestion of the proteins with trypsin, the pH of the obtained solution was adjusted to 8-8.5 with 1 M HCl. After addition of 5 μL 45 mM dl-dithiothreitol (DTT), to reduce sulphur bridges, the solution was mixed and incubated for 30 minutes at 37° C. The solution was cooled down to room temperature, and 5 μL 0.1 M iodoacetamide was added for methylation of the cysteine residues. The solution was mixed on a vortex and incubated for 30 minutes at room temperature in the dark. For protein digestion, 5 μg trypsin in 1 mM HCl (pH<3) was added, followed by 20 μL acetonitrile, mixed by vortex and incubated 1 hour at 37° C. Digestion was stopped by the addition of 0.9 mL 5% formic acid. Then, an internal standard solution containing isotope-labelled bST peptide was added. This peptide is a replicate of the 17 amino acids of the N-terminal end of endogenous bST (AFPAMSLSGLFANAVLR (SEQ ID NO:)) and only differs in the N-terminal amino acid from the rbST incorporation of alanine-13C6 15N4, resulting in a total mass increase of 10 Da. The digest was concentrated on an Agilent Bond Elut Plexa SPE column (60 mg): After conditioning the column with 1 mL methanol and 1 mL 5% formic acid in water, the sample was applied onto the column. Then, the column was washed with 1 mL 10% acetonitrile and the sample was eluted with 0.5 mL water/acetonitrile/formic acid (25:70:5, v/v/v). The eluate was collected in 50 μL DMSO and evaporated to approximately 60 μL on a TurboVap at 55° C. under 10 psi N2. Please note that the exact volume is not critical due to the use of an internal standard. After cooling to room temperature, 25 μL 5% formic acid was added. The sample was mixed and transferred to an LC injection vial. The digestion and concentration procedure took in total 3 hours.

### LC-MS/MS Analysis

Analysis was performed using an I-Class UPLC system connected to a Xevo TQS mass spectrometer Waters (Manchester, UK). Thirty microliters from the final extract was analyzed by UHPLC-MS/MS in multiple reaction monitoring (MRM) mode. The chromatographic separation was performed on a Kinetex 50×2.10 mm I.D. 1.3 μm C18 (100 Å) column. The flow rate was set at 0.5 mL minute-1. A gradient was used starting with 75:25 (v/v) water/acetonitrile for the first 30 seconds, increasing to 70:30 (v/v) water/acetonitrile in the next 3 minutes. Then, the column was washed for half a minute with 100% acetonitrile. Total run time was 6 minutes 30 seconds. The mass spectrometer was operated in the positive ion ESI-MS/MS mode. Ion transitions m/z 913.1>774.1 and m/z 913.1>1047.6 were measured to detect the rbST specific N-terminal peptide with amino acid sequence, MFPAMSLSGLFANAVLR (SEQ ID NO:), after tryptic digestion (Le Breton et al., 2008, Rapid Commun. Mass Spectrom. 22:3130-3136). To check the retention time of this N-terminal rbST peptide of interest, a synthetic analogue of the rbST peptide was injected at the beginning and the end of each series. For the bST internal standard, the transition m/z 888.1>779.13 was followed.

### In-House Method Validation

The decision limit CCα and the detection capability CCβ were determined according to the calibration procedure conform Commission Decision 2002/657/EC. Calculation of the concentration was performed by constructing a linear calibration curve of the response factor (peak area ratio of rbST fragment and internal standard) vs the concentration (expressed as absolute amount rbST protein). For intra-assay variation, four identical rbST spiked serum samples of, respectively, 2 and 10 ng mL-1 rbST in serum were analyzed in parallel. Variation was determined and expressed as the percentage of the average. For determination of inter-assay variation, the spiked serum samples of 2 and 10 ng mL-1 were prepared, enriched with monolith micro-columns and measured on three different days. Variation was determined and expressed as the percentage of the average. For recovery of the immuno-affinity isolation, rbST-spiked serum samples were analyzed and compared to rbST calibration curve in sodium hydroxide, as the latter is compatible with the elution conditions after immune-affinity rbST enrichment.

### Results

Optimization rbST Immuno-Affinity Enrichment

For the enrichment of low abundant rbST from serum of dairy cattle, two monolith micro-columns were compared: a monolith micro-column prepared with protein A and a monolith micro-column prepared with protein A/G. Both protein A and protein A/G have a high affinity for polyclonal rabbit antibodies (Roque et al., 2007, J. Chromatogr. A. 1160:44-55) and are expected to strongly interact with the rabbit anti-rbST used in this study. After rabbit anti-rbST immobilization on both monolith micro-columns, the protein A monolith micro-column was able to capture 10% to 20% more rbST from spiked serum samples compared with the protein A/G monolith micro-column. Therefore, the protein A monolith micro-column was used for further optimization steps.

To obtain the highest recovery of rbST, the number of pipetting cycles, pipetting speed, antibody immobilization concentration and elution conditions were investigated with spiked serum samples. The number of pipetting cycles was determined to be 2000, taking both the efficiency of enrichment and time into consideration. The pipetting speed was found to be of great importance for both immobilization of the antibody on the monolith micro-column and for rbST capture from serum samples. To effectively immobilize the polyclonal anti-rbST antibody onto the monolith micro-column, the solution had to be transferred through the column with slowest pipetting rate as practically possible (approximately 63 μL s-1). Otherwise, immobilization of the antibody was not sufficient and no rbST was captured. The same result was obtained for the transfer of serum samples over the monolith micro-column to enable rbST capture: rbST could only be detected when the serum was transferred slowly through the micro-column (approximately 77 μL s-1).

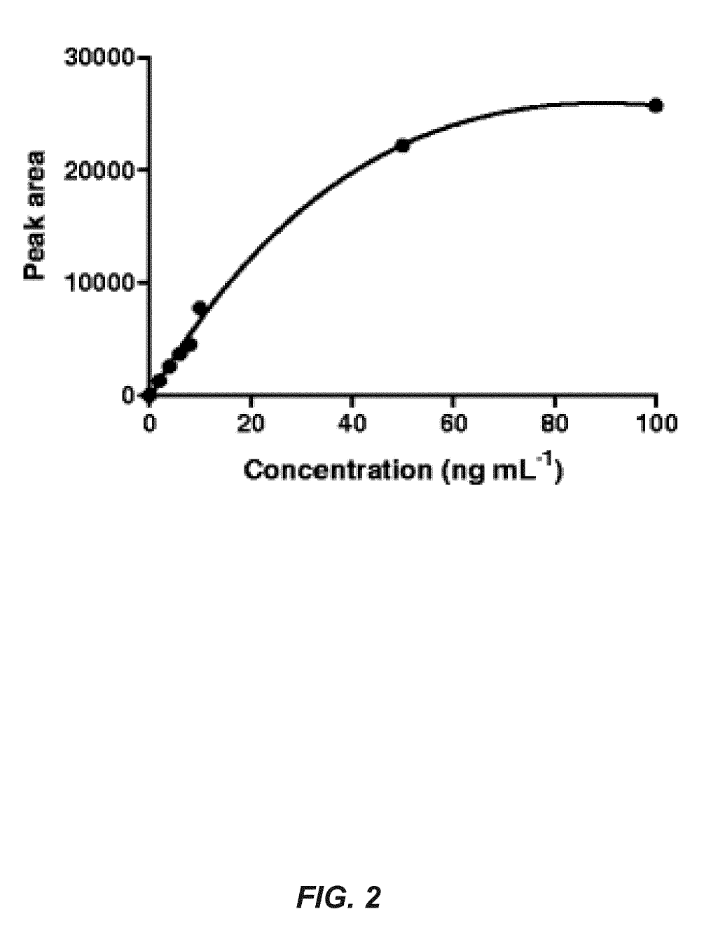

Next, the concentration of anti-rbST antibody used for immobilization to the monolith micro-columns was optimized, aiming for the highest yield of rbST after enrichment. For this, anti-rbST was applied to the column with concentrations of 0.1, 0.07, 0.04 and 0.01 mg mL-1 in 20 mM phosphate buffer pH 7. The immobilization concentration of 0.1 mg mL-1 showed best rbST capture capacity respectively 2, 3 and 7 times more rbST was captured using 0.1 mg mL-1 compared with the other concentrations. Higher concentrations of the antibody were not tested as immobilization with 0.1 mg mL-1 antibody was capable to enrich rbST in serum at concentrations in the low nanogram-per-milliliter range, sensitive enough for incurred serum samples. The binding sites of the micro-columns prepared under these conditions were found to be saturated at a serum rbST concentration of >50 ng mL-1, which is at least fifty times higher than the expected levels of rbST in treated cows (FIG. 2).

For the elution of captured rbST, conditions compatible with the subsequent trypsin digestion are preferred to simplify the workflow. Trypsin digestion compatible buffers are for instance tromethamine (Tris) and ammonium bicarbonate. These buffers were tested in different molarities for their capacity to elute rbST from the monolith micro-columns. Unfortunately, these conditions were too mild, only eluting 10% or less rbST from the monolith micro-columns in comparison with harsher elution conditions. A solution of 200 mM NaOH was found to be most effective for elution of rbST from the micro-columns. After elution, a Tris solution was added to obtain a trypsin compatible solution.

The entire workflow developed is as follows: after enrichment of rbST with monolith micro-columns from serum supernatant, tryptic digestion yields 20 different peptides. The endogenous and recombinant protein differs by one amino acid located at the N-terminal side of the protein. In case of rbST, an alanine is replaced by a methionine. To discriminate between these two forms, the N-terminal peptide is analyzed. To be sure that the detected N-terminal peptide is specific, a blast computation was performed at the SIB using the BLAST network service (Altschul et al., 1997, Nucleic Acids Res. 25:3389-3402). There were no other peptides found containing the same amino acid sequence as the detected peptide. The addition of the isotope-labelled N-terminal peptide of bST as an internal standard enabled correction for sample cleanup losses after tryptic digestion.

### Method Performance Characterization

To characterize the method performance, the developed method was partly validated in-house as a quantitative confirmatory method according to Commission Decision 2002/657/EC (Commission Decision 2002/657/EC of 12 Aug. 2002 implementing Council Directive 96/23/EC concerning the performance of analytical methods and the interpretation of results, Off. J. Eur. Commun. L. 221:8-36). The parameters considered were the decision limit (CCα), the detection capability (CCβ), the intra-assay variation, the inter-assay variation and the recovery. Ion ratio of the measured transition determined for samples from the matrix matched sample (MMS) series and samples from rbST-treated animals were all within the ion ratio limits, as described in the Commission Decision 2002/657/EC.

Important parameters for performance characterization are (i) the decision limit (CCα), the limit at and above which it can be concluded, with an error probability of α, that a sample is non-compliant; (ii) the detection capability (CCβ), the smallest content of the substance that may be detected, identified and quantified in a sample with an error probability of β; (iii) the intra-assay variation; (iv) the inter-assay variation; and (v) the recovery. In the case of substances for which no permitted limit has been established, the detection capability is the lowest concentration at which a method is able to truly detect contaminated samples with a statistical certainty of 1-β.

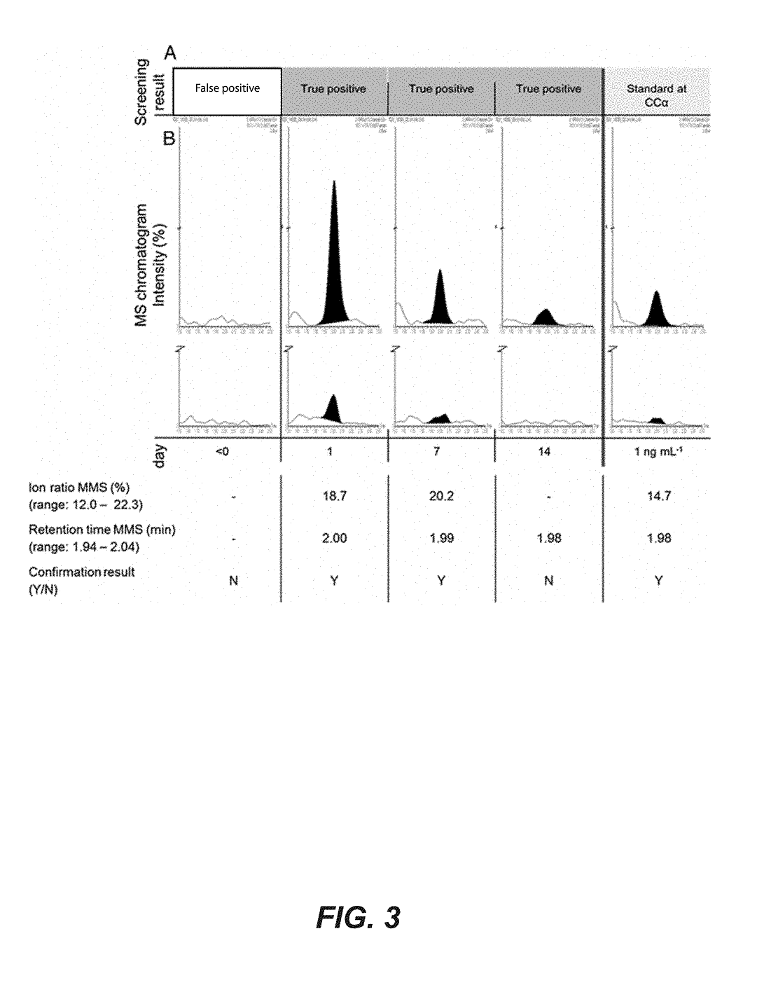

Data obtained from the validation study are presented in Table 1. The CCα and CCβ were determined to be 0.8 and 1.6 ng mL-1, respectively. From additional samples spiked with rbST in concentrations of 0.25, 0.5 and 1 ng mL-1, it was concluded that the obtained CCα and CCβ are realistic, as the 0.5 and 1 ng mL-1 samples still meet the ion ratio criteria (see also the reconstructed ion chromatogram of the rbST-spiked serum sample at 1 ng mL-1 in FIG. 3). Moreover, in 75% of blank serum samples spiked at CCα (0.8 ng mL-1), rbST presence was confirmed, which is actually better than the >50% at CCα level as required by 2002/657/EC.

TABLE 1
In-house validation study to characterize the method performance
using monolith micro-columns loaded with protein A, tryptic
digestion and LC-MS/MS analyses.
Validation results

| | | |
|---|---|---|
| CCα | | 0.8 ng mL⁻¹ |
| CCβ | | 1.6 ng mL⁻¹ |
| Intra-assay variation | 2 ng mL⁻¹ | 12% (n = 4) |
| | 10 ng mL⁻¹ | 9% (n = 3) |
| Inter-assay variation | 2 ng mL⁻¹ | 15% (n = 3) |
| | 10 ng mL⁻¹ | 10% (n = 3) |

Intra- and inter-assay variations were determined for rbST in serum at a concentration of 2 and 10 ng mL-1, in accordance with expected serum concentrations. Variation was expected to be higher at 2 ng mL-1 compared with 10 ng mL-1. Although this difference was indeed observed in both intra- and inter-assay variation, intra-assay variation was found to be only 12% at 2 ng mL-1 and 9% at 10 ng mL-1 rbST in serum (Table 1). It should be noted that for 10 ng mL-1, one of the four data points was removed as outlier due to the fact that the internal standard was not added correctly.

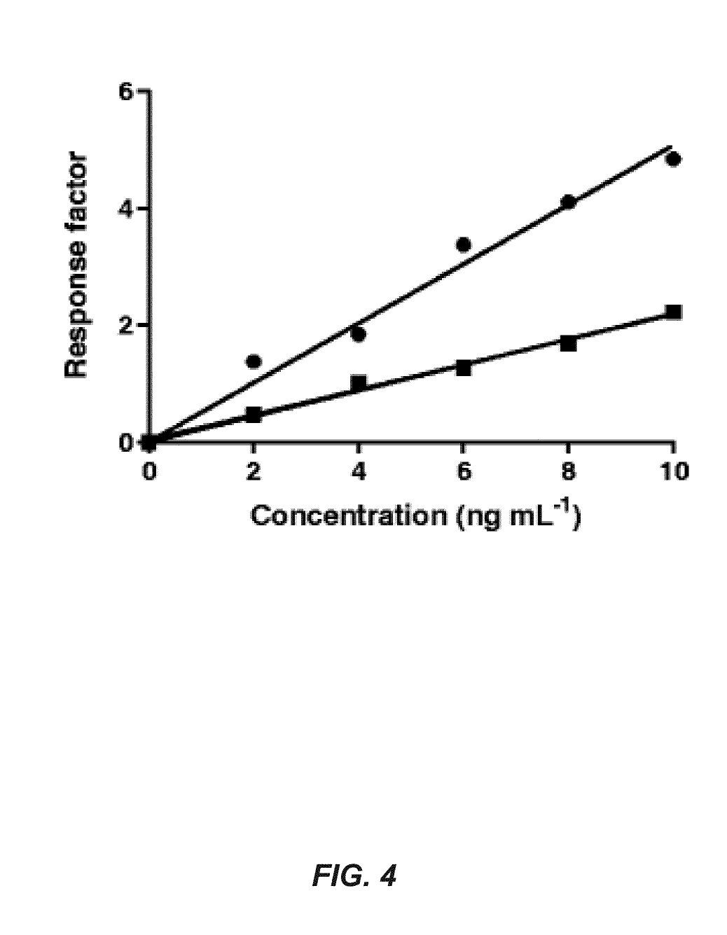

Recovery was determined by comparison of calibration curves prepared in either elution solvent or by fortification of serum. The graphs in FIG. 4 show that not all rbST from the fortified serum samples will be captured and eluted using the monolith micro-columns. Comparison of the response factors of the two calibration curves suggests a recovery during immuno-enrichment of approximately 50% for concentrations up to 10 ng mL-1. For the two highest concentrations, 50 and 100 ng mL-1, the recovery of rbST dropped to 25% and 17%, respectively, due to saturation of the binding sites of the monolith micro-column (FIG. 2). Even though not all rbST is recovered, the repeatability and reproducibility data in Table 1 are fit for purpose and the sensitivity required for effective control is reached.

To test the stability of the trypsin-digested serum, samples were stored for 2 weeks at 4° C.-8° C. Comparison of the samples before and after storage showed a decrease in peak area of approximately 50% for all samples (results not shown). The decrease in intensity of the measured N-terminal peptide can be explained by its instability and tendency to adsorb to glassware (Blokland et al., 2013, in de Almeida et al. (eds) Farm Animal Proteomics 2013, Wageningen Academic Publishers, Wageningen). Although 50% is a significant loss at 4° C.-8° C., it did not obstruct measurement and quantification of rbST in the extracts. It is, therefore, advised to store digested samples and matrix-matched calibrants at 4° C.-8° C. as short as possible prior to analysis.

Detection of rbST in bovine serum has already been presented before by Le Breton et al., 2009, Anal. Chim. Acta. 637:121-127). In that work, the lowest presented rbST concentration in bovine serum was 3 ng mL-1 (data acquired on only one transition). To reach that level, an extensive sample preparation of multiple precipitation steps and overnight digestion was needed. In contrast, the method presented in this study shows high sensitivity (CCα of 0.8 ng mL-1) and the confidence of data acquisition of two ion transitions. Note that using a single-ion transition, rbST in bovine serum with a concentration of 0.25 ng mL-1 could even be detected. Moreover, sample preparation is less extensive, less laborious and semi-automated, and tryptic digestion required only 1 hour.

rbST Analysis in Serum of rbST-Treated Cows

To investigate applicability of the developed method to real-life samples, serum samples from two different animal experiments were analyzed. As a proof of principle, from the first animal experiment, 17 serum samples of one treated cow were analyzed: two serum samples prior to treatment (t=<0 days), one serum sample for each day after the first and second treatment (t=1-14 days) and one sample 3 weeks after the second treatment (t=29 days). This allowed exploring the detectability of rbST during and after the treatment period. Obtained rbST concentrations, corrected for incomplete recovery, are given in Table 2 and show that rbST can be detected from the first day after the first treatment until 7 days after the second treatment. Only in one serum sample, taken on day 2, no rbST seemed to be present. It is not clear why no rbST was detected in the sample that day. The highest concentration of rbST (21.4 ng mL-1) was detected on the first day after the first treatment. On the first day after the second treatment, an increase in rbST was observed as well, although less apparent. In general, a daily variation in rbST concentration was observed and rbST concentrations decreased only slowly. This suggests the ability of the slow-releasing formulae to release rbST slowly to the blood circulation, with a peak on the first day(s), and maintain a minimum level. Twenty-one days after the last treatment, no rbST could be detected anymore, which is in good agreement with the need of a two-weekly treatment, as advised by the manufacturer's (POSILAC®) Supplementation Guide ((elanco.us/pdfs/usdbupos00017-posilac-injection-1-pager.pdf). This treatment schedule was applied in the second animal study. Analysis of samples from this study gives insight in the ability of the method to detect rbST use under realistic treatment conditions. Four serum samples were analyzed: one serum sample prior to treatment (t=<0 days) and serum samples taken after 1 day (t=1 day), 1 week (t=8 days) and 2 weeks (t=14 days) after the third treatment. In addition, three additional blank serum samples from other animals were analyzed.

No rbST was detected in any of the five blank serum samples, and no interferences were observed that could lead to false-positive findings. This implies that the method is very specific. From the treated cow (b), the trend of the determined rbST concentrations is similar to the first animal experiment. The highest concentration was found on the first day after administration, and rbST could be detected until 1 week after administration (see Table 2 and FIG. 3). The difference in concentration on the first day after treatment between the two cows from the two animal experiments can be explained by natural variation in response, which should be investigated by analysis of more treated animals. For the serum sample taken two weeks after rbST administration, just prior to the following administration, a peak is clearly visible at transition m/z 993.1>m/z 774.13. This clearly demonstrates the presence of rbST (FIG. 3). Further enhancement of the sensitivity can be further enhanced by increasing the sample volume. It is expected that by this adjustment, rbST presence can not only be detected but also confirmed up to 14 days after rbST administration. The results compare favorably!! with previous methods (Le Breton et al., 2009, Anal. Chim. Acta. 637:121-127), in which rbST was only detected until 4 days after treatment.

### Confirmation of Positive Screening Results

Control strategies in food and feed safety often include two steps: First, samples are screened in order to obtain a fast indication of suspect samples, thereby reducing the number of samples, as samples with a negative result will not be investigated further. The second step in the control strategy is the confirmation of positive samples conform Commission Decision 2002/657 with additional (analytical) methods (van den Broek et al., 2013, Journal of Chromatography B 929:161-179). This two-step strategy was applied to the four samples from the second animal study. The samples were previously screened by Ludwig et al., 2012, PLoS ONE 7:e52917 by a multiple protein biomarker assay, where four different biomarker proteins were measured simultaneously. The serum samples were selected for analysis with the LC-MS/MS method as they were considered suspicious for rbST in the screening assay (FIG. 3a). The sample taken prior to treatment (t=<0 days) was screened suspect, which is obviously unlikely and might serve as a false-positive case. Analysis of the serum sample prior to treatment (t=<0 days) with the confirmatory method showed that, indeed, despite the positive screening result, no rbST-specific peptide could be detected and rbST was below the detection limit (FIG. 3b). It can, therefore, be concluded that the screening result of this sample, taken before treatment, is false positive and underlines the necessity of confirmatory methods. The false-positive screening result was most likely due to the apparent presence of rbST-induced antibodies as was observed in less than 5% of the untreated cows and is most likely the result of non-specific interactions of other antibodies or proteins in the screening assay (Smits et al., 2012, Drug Test Anal. 4:362-367).

For the other three serum samples, taken during rbST treatment according to the treatment schedule, screening results were found to be true positive. As shown in FIG. 3b by the chromatograms of transition m/z 913.1>m/z 774.13 and m/z 913.1>m/z 1047.6, samples taken during the rbST treatment all showed rbST presence.

Samples taken after treatment of dairy cows with rbST (t=>0 days), which were found positive in the screenings assay, were confirmed for the presence of rbST with the developed confirmatory LC-MS/MS method (FIG. 3). In addition, the need of a reliable confirmatory method for samples found positive during screening was proven by the example of a false-positive screening result that could only be identified by the LC-MS/MS analysis. Please note that no explicit confirmation criteria have been established yet for protein and/or peptide analysis by targeted MS/MS.

TABLE 2
RbST concentrations found in serum samples of two dairy
cows, a and b treated with rbST, quantified versus MMS.

| | Cow | |
|---|---|---|
| | a | b |
| | Concentration rbST | Concentration rbST |
| Day | (ng mL⁻¹) | (ng mL⁻¹) |

| | | | | |
|---|---|---|---|---|
| <0 | | <CCα | <CCα | |
| | [symbol] | | | [symbol] |
| 1 | | 20.3 | 9.0 | |
| 2 | | <CCα | — | |
| 3 | | 4.8 | — | |
| 4 | | 4.5 | — | |
| 5 | | 6.7 | — | |
| 6 | | 2.3 | — | |
| 7 | | 1.4 | — | |
| | [symbol] | | | |
| 8 | | 1.8 | 1.9 | |
| 9 | | 9.1 | — | |
| 10 | | 3.0 | — | |
| 11 | | 2.8 | — | |
| 12 | | 1.8 | — | |
| 13 | | 4.0 | — | |
| 14 | | 2.1 | <CCα | |
| 29 | | <CCα | — | |

“Syringes” - (left side for cow a; right side for cow b) show days of treatment with rbST in slow releasing formulae
“—” not determined

### Example 2. Indirect Detection

### Materials and Methods

### Chemicals and Instruments

Ultrasonic bath, monosodium phosphate monohydrate (NaH₂PO₄×H₂O), potassium dihydrogen phosphate (KH₂PO₄), sodium chloride (NaCl), sodium azide (NaN₃) and TWEEN®-20 were obtained from VWR International (Amsterdam, The Netherlands). Microcentrifuge Model 16K was purchased from Bio-Rad (Veenendaal, The Netherlands). Protein LoBind Tubes, Safe Lock Tubes (amber) and Centrifuge 5810R were obtained from Eppendorf (Hamburg, Germany). 1-Ethyl-3-(3-dimethylaminopropyl)carbodiimide (EDC), 2-(N-morpholino)ethanesulfonic acid (MES) hydrate, ovalbumin and bovine serum albumin (BSA) were obtained from Sigma-Aldrich (St. Louis, Mo., USA). MultiScreen HTS filter plates were purchased from Millipore (Billerica, Mass., USA). Purified bovine osteocalcin and mouse anti-bovine osteocalcin antibodies were obtained from Haematologic Technologies, Inc. (Essex Junction, Vt., USA). Insulin-like growth factor-I (IGF-I; human recombinant) was purchased from Fitzgerald Industries International (North Acton, Mass., USA). Insulin-like growth factor binding protein-2 (IGFBP-2; bovine recombinant, receptor grade) was purchased from IBT (Reutlingen, Germany). Mouse anti-IGF-I was supplied by LifeSpan BioSciences, Inc. (clone SPM406, Seattle, Wash., USA) and the rabbit anti-IGFBP-2 was from United States Biological (Swampscott, Mass., USA). Monsanto rbST standard was obtained from the National Hormone & Peptide Program (NHPP) of Dr. Parlow (Torrance, Calif., USA). R-Phycoerythrin (PE)-labelled goat anti-bovine immunoglobulins (GAB-PE) were from Santa Cruz Biotechnology (Santa Cruz, Calif., USA) and R-Phycoerythrin (PE)-labelled goat anti-mouse immunoglobulins (GAM-PE) and goat anti-rabbit immunoglobulins (GAR-PE) were purchased at Prozyme (San Leandro, Calif., USA). Donor adult bovine serum was from HyClone (South Logan, Utah, USA). Sodium hydroxide (NaOH), disodium hydrogen phosphate dihydrate (Na₂HPO₄×2 H₂O) and hydrochloric acid (HCl) were purchased from Merck (Darmstadt, Germany). SeroMAP microspheres (microsphere sets 025, 050, 078 and 084) and sheath fluid were obtained from Luminex (Austin, Tex., USA). The Luminex 100 IS 2.2 system consisting of a Luminex 100 analyzer and a Luminex XY Platform was purchased from Applied Cytometry Systems (ACS, Dinnington, Sheffield, South Yorkshire, UK). Snijder Test tube rotator was from Omnilabo International (Breda, The Netherlands). 10 mL polypropylene tubes were obtained from Greiner Bio-One (Alphen aan de Rijn, The Netherlands). Glycine was purchased from Duchefa (Haarlem, The Netherlands) and sulfo-N-Hydroxysuccinimide (Sulfo-NHS) from Fluka (Buchs, Switzerland). Sodium dodecyl sulphate (SDS) was obtained from Serva (Heidelberg, Germany). The microtiter vari-shaker was purchased from Dynatech (Guernsey, UK). POSILAC® (rbST) 500 mg single dose syringes and syringes with only the slow release formula were obtained from Monsanto Company (St. Louis, Mo., USA) for animal study I and from Elanco Animal Health (Greenfield, Ind., USA) for animal study II.

### Buffers and Solutions

Buffers and solutions were prepared as follows: phosphate-buffered saline (PBS; 154 mM NaCl, 5.39 mM Na₂HPO₄, 1.29 mM KH₂PO₄, pH 7.4), PBST (PBS, 0.05% v/v TWEEN®-20), PBSTB (0.1% w/v BSA in PBST), glycine solution I (GS 1; 27.5 mM glycine, pH 0.5 adjusted with HCl), glycine solution 11 (GS II; 400 mM glycine, 0.3% w/v SDS, pH 10 adjusted with NaOH), MES buffer (50 mM, pH 5), blocking buffer (PBS, 0.1% w/v BSA, 0.02% v/v TWEEN®-20, 0.05% w/v NaN₃).

### Sample Materials

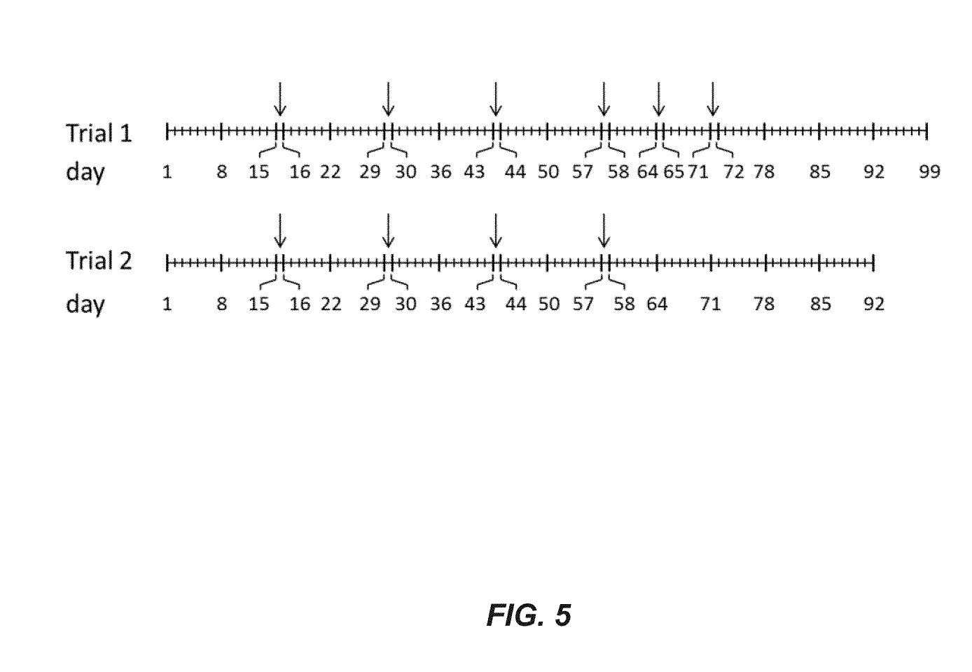

Samples from different sources were used for analysis. Serum samples from two independent controlled animal treatment studies were used. In animal study I, eight Holstein cows were selected. These cows were all about 5 years old, divided in two groups of four animals each and treated with 500 mg rbST in slow-release formula or slow-release formula only. After a two-week adaptation period, they received an injection every second week, in total four times in accordance with the suggested treatment schedule by the manufacturer (http.//www.fda.gov/downloads/AnimalVeterinary/Products/ApprovedAnimalDrugProducts/FOI ADrugSummaries/ucm050022.pdf; accessed 2012 Apr. 4). Since it was not known for sure whether any response would be seen, the cows were thereafter treated two more times with a weekly interval, followed by a final four-week withdrawal period. In animal study II, 10 Holstein dairy cows were divided in two groups. In contrast to animal study I, these cows were of different age (2-8 years). After a two-week adaptation period, eight cows were treated every second week with 500 mg rbST in a slow-release formula during eight weeks and two control cows were treated with the slow-release formula only. The biweekly treatment period according to manufacturers' guidelines was directly followed by a four-week withdrawal period. In both studies, blood sampling was scheduled similarly: During an adaptation period of two weeks, blood samples were collected weekly; during the treatment period, blood samples were collected a day before, a day after and a week after injection and during withdrawal, blood samples were collected weekly for four more weeks, which yielded 21 serum samples per cow in animal study I and 18 serum samples per cow in animal study II. The treatment schedule and blood sampling time points are shown in FIG. 5. Unfortunately, one untreated cow died in the beginning of animal study I because of swollen hocks, which led to general inflammation and sepsis. Therefore, results could be obtained for four rbST-treated and three untreated cows in study 1. Furthermore, one cow from animal study II fell sick (hock joint inflammation, lung infection and sepsis) in course of the experiment and its biomarker level results were excluded from statistical analysis. For investigation of natural physiological variations in biomarker levels, sera from 67 healthy, lactating cows varying in the age of two to eleven years, from two different locations, in different stages of their lactating cycle were analyzed, to reflect a normal population of untreated dairy cows. Based on the origin of these animals the assumption of being untreated with rbST was justified.

### Standard Preparation

Protein standards of IGF-1, IGFBP2 and osteocalcin, prepared in serum-matched buffer (80 mg mL⁻¹ BSA in PBS), were used for standard curves ranging from 0.08 to 20 ng mL⁻¹ for IGF-I and osteocalcin and from 0.2 to 50 ng mL⁻¹ for IGFBP2. Also blank standard samples (80 mg mL⁻¹ BSA in PBS without any IGF-1, IGFBP2 and osteocalcin) were measured. Note that no standards are commercially available for anti-rbST-antibodies.

### Sample Pre-Treatment

A generic sample pre-treatment procedure which was crucial for removing non-specific interferences and making the candidate biomarkers accessible for detection was described previously (Smits et al., 2012, Drug Test Analysis 4:362-367; Bremer et al., 2010, Analyst 135:1147-1152; Ludwig et al., 2012, Food Control 26:68-72).

After blood collection, blood samples were kept at room temperature for 4 hours to coagulate. After coagulation, samples were centrifuged for 10 minutes at 3,000 g, and sera were collected. Serum samples were stored at −80° C.

For the generic serum pretreatment procedure 25 μl glycine solution 1 (27.5 mM glycine pH 0.5) were added to 25 μl serum or standard sample in a polypropylene tube under constant vortexing. After 60 minutes incubation at room temperature, 50 μl glycine solution II (400 mM Glycine, 0.3% m/v SDS, pH 10) were added under constant vortexing and samples were further diluted by addition of 1.9 mL 0.1% BSA in PBST (in total, 80 times diluted).

### Microsphere Preparation

Covalent coupling of 100 μg mL⁻¹ Monsanto rbST standard, 100 μg mL⁻¹ IGF-1 and 10 μg mL⁻¹ IGFBP2 to seroMAP microspheres (sets 050, 025, 078, respectively) was described before (Smits et al., 2012, Drug Test Analysis 4:362-367; Bremer et al., 2010, Analyst 135:1147-1152; Ludwig et al., 2012, Food Control 26:68-72). Coupling 75 μg mL⁻¹ osteocalcin to microspheres (set 084) was done following the same procedure.

### Four-Plex Flow Cytometric Immunoassay Procedure

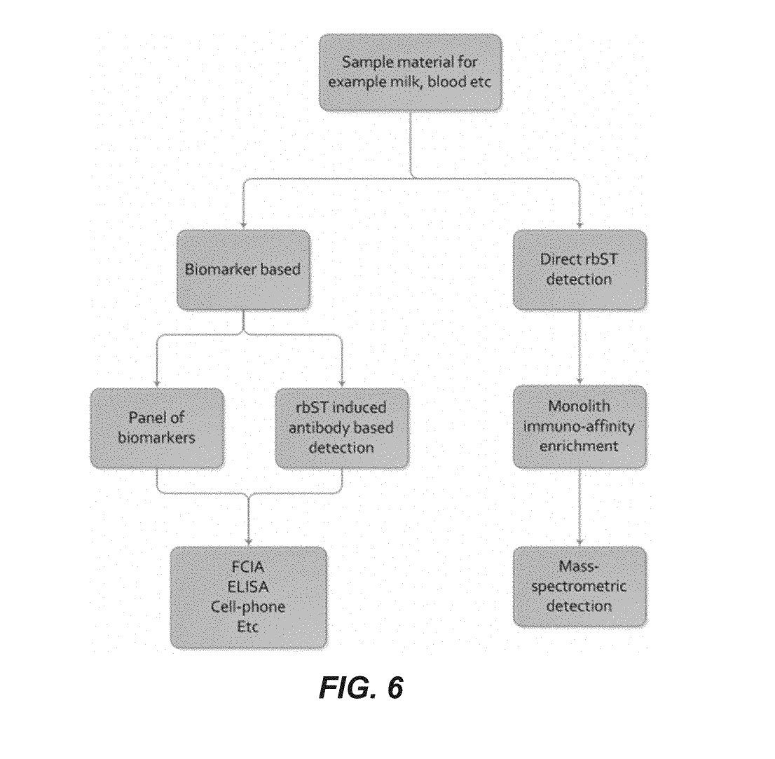

An assay procedure for detection of three biomarkers was described before (Smits et al., 2012, Analyst 138:111-117) and is similar for four biomarkers in the present study and summarized in FIG. 6. The samples were analyzed in duplicate in the flow cytometer at 1 μL s⁻¹ until 50 microspheres per set were counted, up to a maximum of 50 μL per sample. A typical analysis of a full 96-well microtiter plate takes 3.5 hours starting from raw serum until the results are obtained.

### Data Analysis

Raw median fluorescence intensities (MFIs) were measured by the flow cytometer for every single candidate biomarker. Every sample was measured in duplicate and MFIs were averaged before further analysis. For IGF-1, IGFBP2 and osteocalcin, B/B0 values were calculated per sample by dividing the measured MFI by the MFI of a blank biomarker-free standard. Then, concentrations were recalculated from standard curves (non-linear four-parameter curve fit) using GraphPad Prism program (GraphPad Software Inc., San Diego, USA) for IGF-1 and osteocalcin. For IGFBP2, no complete inhibition could be obtained with the available standard protein, therefore, no actual concentrations were determined and B/B0 values were simply used. For anti-rbST-antibodies, which are endogenously produced by the cow in response to rbST treatment, no standard was available. To be able to normalize, measured sample MFIs were divided by the MFI of one serum sample, which was measured every time (B/Bd). This serum was donor adult bovine serum which was a mixture of sera from many cows from one herd. Since this is produced in large amounts, it can be used for a long time with constant quality.

To assess the four-plex FCIA quality and compare it to other methods, assay performance characteristics were calculated, such as IC₅₀, inter-assay and intra-assay variation (describing precision and ruggedness). For IGF-1, IGFBP2 and osteocalcin, IC₅₀ was read from standard curves at 50% inhibition of the signal of the blank. For all candidate biomarkers, inter-assay variation was determined by measuring eight different serum samples on 8 days. Mean of results (concentrations for IGF-1 and osteocalcin, B/B0 for IGFBP2 and B/Bd for anti-rbST-antibodies), standard deviation and percentaged standard deviation (% CV) were calculated for every serum. The average of the eight percentaged standard deviations was the inter-assay deviation. Intra-assay variation was calculated the same way from eight repetitions of eight sera within one microtiter plate.

### Single Biomarker Analysis Approach

Using a single biomarker for prediction of unknown samples as rbST-treated or untreated, the calculation of decision limits for each biomarker was necessary. These were based on the results obtained from a population of 67 untreated dairy cows being diverse in age, in lactation stage and in origin. For every biomarker, results were averaged and two times the standard deviation was added to obtain the decision limit. Samples found to show concentrations (for IGF-1 and osteocalcin), B/B0 (for IGFBP2) or B/Bd (for anti-rbST antibodies) beyond the respective calculated decision limit, were considered as rbST-treated (positive). True-positive and false-positive rates could be calculated for every single biomarker from the results of the controlled animal studies.

### Additive Biomarker Analysis

After evaluating biomarker profiles and true-positive rates based on single biomarkers, an additive biomarker approach was tested. Here, a sample was considered as rbST-treated when at least one of the candidate biomarkers reacted above decision limit and also here, true-positive and false-positive rates were calculated.

### Multiple Biomarker Statistical Approach

After evaluating single candidate biomarkers and testing the additive biomarker approach, how well a statistical combination of two to four markers was capable of predicting rbST abuse was assessed. Therefore, a k-nearest neighbors prediction model (kNN) in the R environment (R. C. Team (2014), RA language and environment for statistical computing, Vienna: R Foundation for Statistical Computing) and functions available in R package e1071 (Dimitriadou et al., 2008, T U Wien, R package 1:5-24), were used to evaluate all eleven theoretical combinations of two to four biomarkers. As in the single biomarker approach, recalculated concentrations for IGF-1 and osteocalcin and B/B0 signals for IGFBP2 as well as B/Bd signals for rbST-induced antibodies for every sample from the animal studies were included in the data analysis. For obvious ethical reasons, only a limited number of rbST-treated animals were available. Therefore, all serum sample time points per cow (21 time points in the trial period of 14 weeks for animal study I and 18 time points in the trial period of 13 weeks for animal study II) were used for data analysis, despite the fact that these were not completely independent. However, only data from independent cows were used for model building and sample prediction.

First, the whole data set was divided into two groups: Group A data were used to build the time-point-independent prediction model. Therefore, and to use sufficient sample numbers for the model building, this group contained all data from animal study II (diverse population with biweekly treatment only). Furthermore, since two control animals were not enough to represent untreated cows, Group A also contained the data from the untreated animals of animal study I. In total, 98 samples from treated and 119 samples from untreated cows were used for model building. Group B data were used for prediction based on the Group A model. Group B contained the data from the rbST-treated cows of animal study I (uniform in age with biweekly treatment and two additional weekly injections) and the 67 untreated cows. Note that these are sample data independent from Group A data.

For model building of the Group A data, a training and test set were chosen by using a stratified repeated random sub-sampling approach, which means that 70% of the rbST-treated and 70% of the untreated samples were selected for the training set and the remaining 30% of both groups for the test set for internal validation, which is necessary to build a strong model. Subsequently, concentrations, B/B0 and B/Bd values of the training set were auto-scaled and a kNN model was built on the training set data. The optimal number of k (1≦k≦10) was chosen based on the bootstrapping approach (Efron, 1979, Ann. Stat. 7:1-26) leaving out 10% of the training data (randomly with replacement), which was repeated ten times. The resulting model was validated with the test set data and thereafter used for predicting Group B data. To obtain an average performance of the model, this procedure was run 10,000 times; every time different randomly chosen training and test sets of Group A data were applied. Correctly and falsely predicted results were evaluated for Group B and a true-positive rate and false-positive rate could be calculated for every Group B sample.

### Results

For the prediction of rbST abuse in dairy cows, candidate biomarkers were selected based on information found in literature (see Table 1 in Ludwig et al., 2012, PLoS ONE 7(12):e52917). These were markers of the IGF-axis (such as IGF-1 and IGFBP2) and bone markers (such as osteocalcin), known to be influenced by somatotropin and previously examined by the GH-2000 group for detecting somatotropin abuse in athletes (Longobardi et al., 2000, J. Clin. Endocrinol. Metab. 85:1505-1512; Kicman et al., 1997, Clin. Endocrinol. 47:43-50). Furthermore, the immune response of cows treated with rbST was examined thoroughly and the presence of the specific endogenous antibodies were used against rbST as a biomarker for its abuse (Smits et al., 2012, Drug Test Analysis 4:362-367; Rochereau-Roulet et al., 2011, Anal. Chim. Acta. 700:189-193; Eppard et al., 1992, J. Dairy Sci. 75:2959-2967; Zwickl et al., 1990, J. Dairy Sci. 73:2888-2895). Although PIIINP, a marker of collagen turnover, is known to show potential in human and bovine hormone abuse detection (Mooney et al., 2008, Biomarkers 13:246-256; Wallace et al., 2000, J. Clin. Endocrinol. Metab. 85:124-133), it has not been included into the biomarker panel yet because of the lack of a suitable commercially available standard protein and antibody.

### Development of a Four-Plex Flow Cytometric Immunoassay

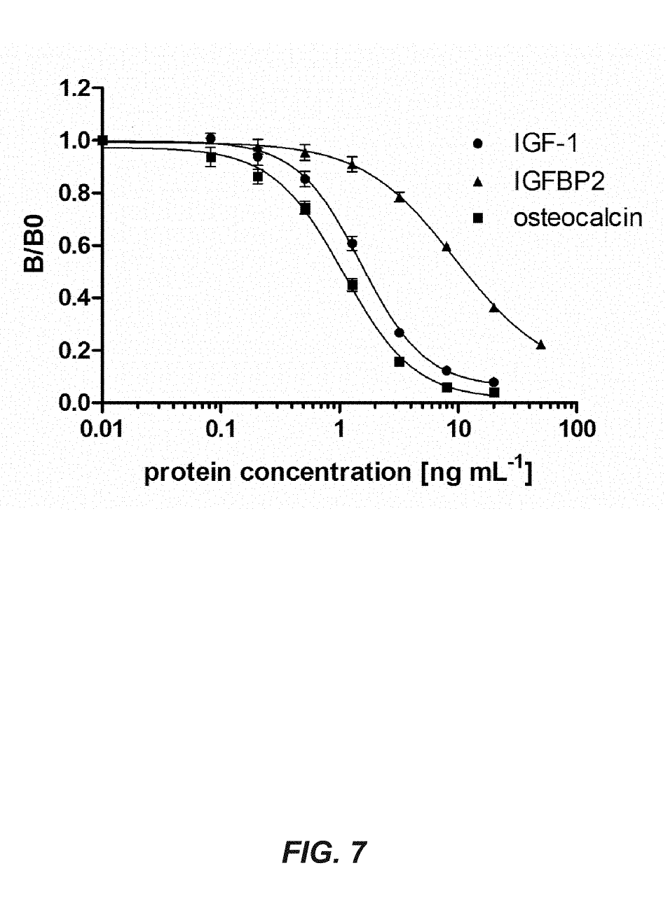

For the simultaneous detection of these four candidate biomarkers, a generic sample pre-treatment and four-plex flow cytometric immunoassay (FCIA) were developed. To this end, the previously reported three-plex assay (Smits et al., 2013, Analyst 138:111-117) was extended with the biomarker osteocalcin. Adding osteocalcin to the existing triplex FCIA did not result in major interferences of any of the assay components of the four combined biomarker assays (data not shown). IGF-I and osteocalcin concentrations of tested serum samples were calculated based on the obtained standard curves in serum-matched buffer (see FIG. 7). The four-plex FCIA is capable of determining IGF-1 and osteocalcin concentrations in the relevant range in serum, namely 64-400 ng mL⁻¹ for IGF-I and 32-320 ng mL⁻¹ for osteocalcin (note that serum samples were diluted 80 times prior to analysis, thus the standard curves cover protein concentrations of 0.8-5 ng mL⁻¹ for IGF-1 and 0.4-4 ng mL⁻¹ for osteocalcin). For IGFBP2, the standard protein could not completely inhibit the B0 signal; therefore, it was decided to work with normalized responses (B/B0) for the data analysis. For the induced anti-rbST-antibodies, the responses normalized to a single standard serum (B/Bd) were utilized.

The generic sample pre-treatment was necessary for releasing IGF-1 from its binding protein-complex and preventing non-specific binding in the detection of anti-rbST-antibodies. The rather harsh pre-treatment protocol did not affect the detection quality of osteocalcin, thus it could be adopted for the combined four-plex FCIA. Note that adding IGF-2 in excess, as done in commercially available human IGF-1 immunoassays, improved neither the normalized standard curves nor the detection of biomarker level differences in between treated and untreated animals. The developed assay showed high reproducibility for all measured candidate biomarkers (Table 3) and a comparable sensitivity to previous single biomarker methods (Lee et al., 2000, Ann. Clin. Biochem. 37:432-446; Armstrong et al., 1993, Domest. Anim. Endocrin. 10:315-324). However, the newly developed four-plex FCIA has several advantages, such as the simultaneous measurement of all four markers in one sample from one well of a microtiter plate, which saves sample material, work load and time. Additionally, only one washing step was required compared to an average of six washing steps in a conventional enzyme-linked immunosorbent assay, making the four-plex FCIA much faster and easy-to-use. The whole assay procedure, starting from a serum sample until the results from the flow cytometer for all four candidate biomarkers, takes 3.5 hours for a whole 96-well microtiter plate. This demonstrates that the four-plex FCIA is a rapid and promising screening tool for the detection of the four candidate biomarkers in serum.

TABLE 3
Four-plex FCIA assay performance characteristics for the single
candidate biomarkers.

| | Candidate biomarkers | | | |
|---|---|---|---|---|
| Performance | | | Anti- | |
| characteristics | IGF-1 | IGFBP2 | rbST antibodies | Osteocalcin |
| IC₅₀ | 1.5^{a} | 9.5^{a} | — | 1.1^{a} |
| Inter-assay variation | 15.7^{b} | 7.9^{b} | 22.3^{b} | 17.1^{b} |
| Intra-assay variation | 6.4^{b} | 5.7^{b} | 9.4^{b} | 9.5^{b} |
| Decision limit | 216^{a} | 0.52^{c} | 1.62^{d} | 160^{a} |

| | |
|---|---|
| Stability | The 4-plex FCIA can be performed stably over |
| | several months by different staff. |
| Specificity | No unwanted interaction in between the assays |
| | (analytes and antibodies) observed. |

IC50 related to 80-times diluted samples
^{a}in ng mL⁻¹.
^{b}in %.
^{c}of B/B0.
^{d}of B/bd.
doi: 10.1371/journal.pone.0052917.t002

Single Candidate Biomarker Profiles of Untreated and of rbST-Treated Cows

After successful development of the four-plex flow cytometric immunoassay, decision limits for each single candidate biomarker were calculated by analysis of sera from 67 untreated dairy cows (see paragraph 9.1 in the materials and methods section). Compared to the number of tested athletes in human studies, the number of tested control animals may seem to be rather low, but the variation within the dairy population is expected to be much lower, because of several reasons: First, only female cows have to be taken into account. Second, milking only occurs after first calving (usually at 20-24 months of age), thus after puberty, in which levels of IGF-1, IGFBP2 and osteocalcin are mainly changed due to growth and are more stable thereafter (Blum et al., 1993, Growth Regulat. 3:100-104; Friedrich et al., 2008, Growth Horm. IGF Res. 18:228-237; Sato et al., 2011, Res. Vet. Sci. 91:196-198). Third, since in this region of Europe mainly Frisian Holstein cows are used for milk production, the focus was on this particular breed for the development of the test. And fourth, no consideration was made for sick animals, since their milk will not be allowed for consumption due to the presence of veterinary drug residues and, therefore, treatment with rbST is useless for sick dairy cows. Thus, the overall relative variation expected in dairy cows is much lower than in athletes, where gender, different ethnicities, the effect of sports discipline, injury and all age groups need to be considered.

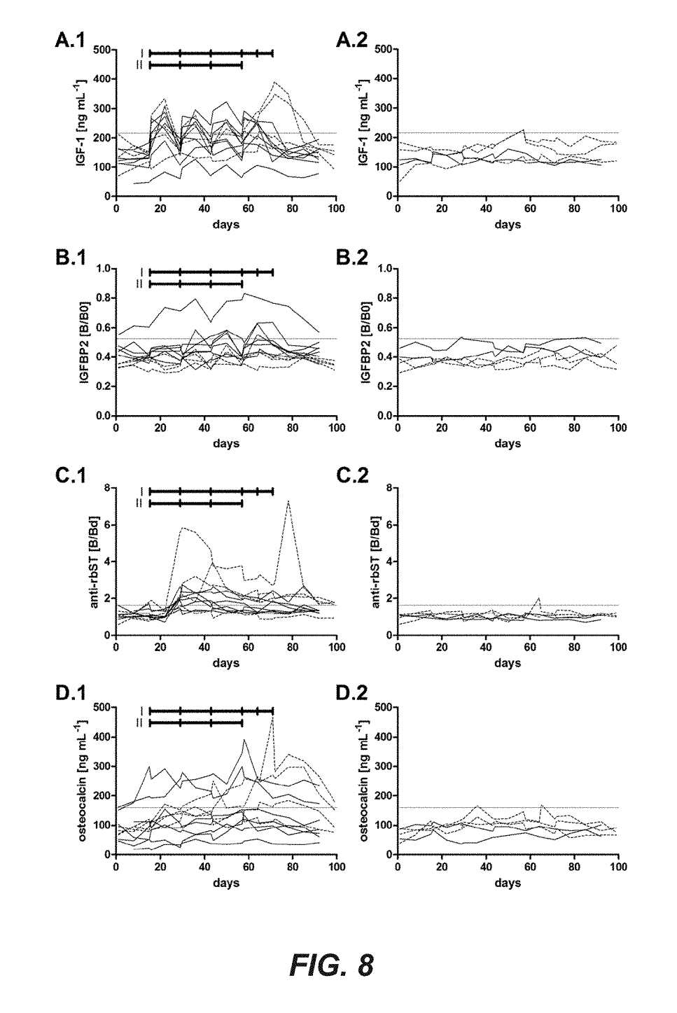

Decision limits were 216 ng mL⁻¹ for IGF-1, 0.52 B/B0 for IGFBP2, 1.62 B/Bd for anti-rbST-antibodies and 160 ng mL⁻¹ for osteocalcin and are shown as horizontal lines in FIG. 8. Results of samples exceeding this limit were considered positive.

Then, biomarker profiles of the dairy cows from both animal studies were measured (FIG. 8). Results of the cows from animal study I are shown in dotted lines whereas the results of animal study II are shown in solid lines. Note that the animals from animal study I received two additional weekly rbST injections after the biweekly treatment period (the treatment schedules of both animal studies are indicated by the black horizontal bars above the graphs and shown in FIG. 8).

IGF-1 levels were found to be elevated directly after rbST treatment (FIG. 8A.1) and returned back to baseline before the next treatment. This short response time was observed before in human studies, where IGF-1 concentrations were back to baseline one week after termination of somatotropin treatment (Kicman et al., 1997, Clin. Endocrinol. 47:43-50). Nevertheless, in athletes, IGF-1 stayed elevated throughout the treatment period. This difference in IGF-I response to somatotropin treatment could be due to the fact, that athletes were injected daily and, although a slow-release formulation was used in the here presented study, the biweekly treatment schedule does not reflect the same situation of permanently present somatotropin in circulation. IGF-1 levels of untreated animals (FIG. 8A.2) remained below the decision limit. The found IGF-1 concentrations are consistent with previously reported serum IGF-1 concentrations in dairy cows (Smits et al., 2013, Analyst 138:111-117; Kerr et al., 1991, Can. J. Anim. Sci. 71:695-705).

IGFBP2 levels (FIG. 8B) are expected to decrease upon rbST treatment (Kicman et al., 1997, Clin. Endocrinol. 47:43-50; Sharma et al., 1994, J. Dairy Sci. 77:2232-2241). The IGFBP2 assay is of an inhibition format, thus B/B0 levels are inversely correlated with the concentration. Hence, higher B/B0 levels are expected after rbST treatment. For some of the rbST-treated cows, a slight increase in B/B0 levels can be observed after treatment (FIG. 8B.1) with a decrease to baseline before the next treatment. But this pattern is not as pronounced as for IGF-I. Furthermore, only occasionally a value exceeded the decision limit. Only the results of one cow were clearly above the decision limit, but these values were observed already during the adaptation period. In humans and despite large inter-individual differences, mean IGFBP2 levels responded quite well upon ST treatment, but the athletes were treated daily on three subsequent days (Kicman et al., 1997, Clin. Endocrinol. 47:43-50). B/B0 levels of untreated animals (FIG. 8B.2) remained below the decision limit at almost all times.

For the antibodies, endogenously produced by the cow as an immunological response upon rbST treatment (Rochereau-Roulet et al., 2011, Anal. Chim. Acta. 700:189-193), a delayed increase in signal was observed (FIG. 8C.1). Most of the cows developed antibodies approximately two weeks after the first rbST injection and a maximum in response could be seen around the third injection (four weeks after start of rbST treatment). Thereafter, the responses declined slowly. Zwickl et al. reported an increase of antibody formation within the first three months of rbST treatment and a decline thereafter, but the amount of rbST administered in their study was much higher than recommended by the manufacturer and applied here (Zwickl et al., 1990, J. Dairy Sci. 73:2888-2895). For the untreated cows in the studies (FIG. 8C.2), only one result was found to be above the decision limit.

For osteocalcin, a slow increase in concentration was observed after rbST treatment (FIG. 8D.1) compared to the untreated cows where the concentrations remained below the decision limit at almost all times. A similar effect on osteocalcin levels was observed in the human GH-2000 study (Longobardi et al., 2000, J. Clin. Endocrinol. Metab. 85:1505-1512). Osteocalcin concentrations in the studies increased consistently in the eight-week treatment period, no gradual decline was observed as for the anti-rbST-antibodies. A slow osteocalcin decrease was noticed after rbST withdrawal but values remained above the decision limit for some of the cows until the end of the animal study.

For all of the candidate biomarkers, large inter-individual physiological differences in biomarker levels were apparent as, for example, seen in the adaptation period of the treated animals. IGF-1, IGFBP2 and osteocalcin levels differed quite a lot between individual animals. Biomarker levels are known to be influenced by many factors such as age and state of lactation. Nevertheless, the expected variation is much smaller than in athletes tested for ST abuse as already discussed above. Note that the variation in the untreated reference population used to assess the decision limits was accounted for. Also, the response upon rbST treatment differed in every individual cow. Some cows showed a big increase in IGF-1 levels short after injection while others did not show any response above decision limit (non-responders). Also for osteocalcin, some cows hardly showed any response after treatment.

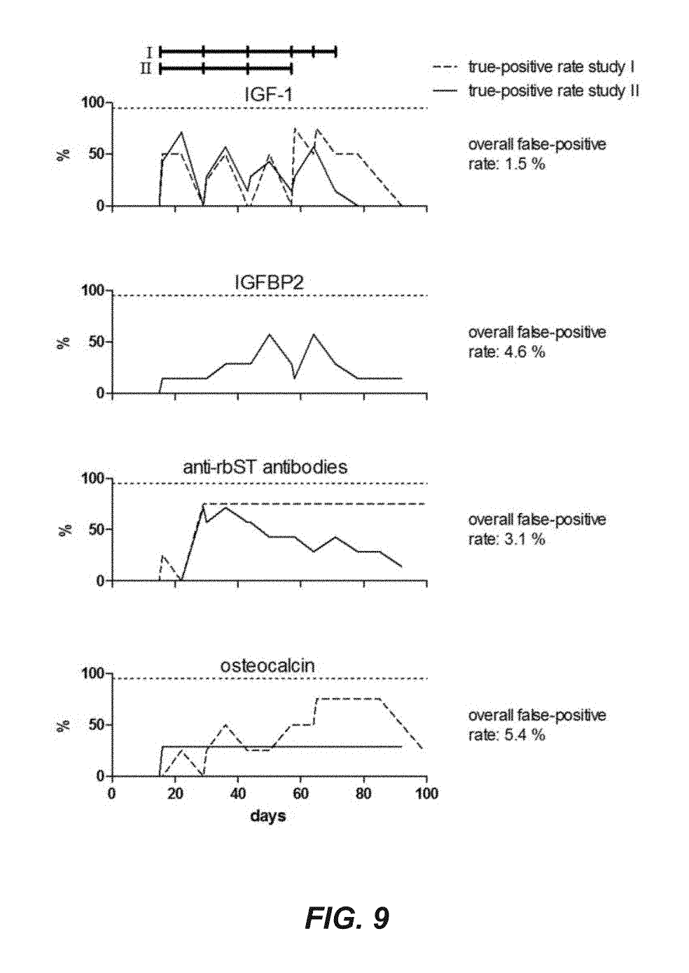

The predictive power of each candidate biomarker was assessed by calculating true-positive rates for all samples from rbST-treated cows in their treatment and withdrawal period (FIG. 9). False-positive rates were calculated from untreated cows during the whole animal experiment (adaptation period samples from all cows and all the samples from untreated cows). High true-positive rates were reached by IGF-1 already at the beginning of the treatment period. Similar response patterns were observed for both studies. Only the double injections in study I led to a changed IGF-1 pattern. Also for the anti-rbST-antibodies, high true-positive rates of 75% were seen after the second rbST injection. But the response was study-dependent: while the animals from study I (equal age of five years) were found positive after the second injection until the end of the study period, a gradual decrease of the number of positively found animals was observed in study II (age ranged from two to eight years). This could be due to the different ages of the animals in study II. It was observed that the antibody response tended to be higher in the older animals. Younger animals also showed antibody response, which declined more quickly than for the older animals. For osteocalcin, as already seen in FIG. 8D.1, some of the rbST-treated cows did not show osteocalcin concentrations beyond the decision limit in both studies. The increase of true-positive found samples at the end of the treatment period in study I was due to the double rbST injections. As already expected from the biomarker profiles (FIG. 8B.1), IGFBP2 did not show high true-positive rates, i.e., none of the animals from study I and only some animals in study II were found above the decision limit. For all of the candidate biomarkers, false-positive rates were quite low, indicating a high specificity of all of the biomarkers towards rbST treatment. Nevertheless, none of the candidate biomarkers reached the targeted 95% true-prediction (<5% false-compliant) rate at any time point required for a screening method according to Commission Decision 2002/657/EC (European Union (2002) Commission Decision 2002/657/EC, Off. J. Eur. Commun. L. 221:8-36).

### Additive Biomarker Analysis

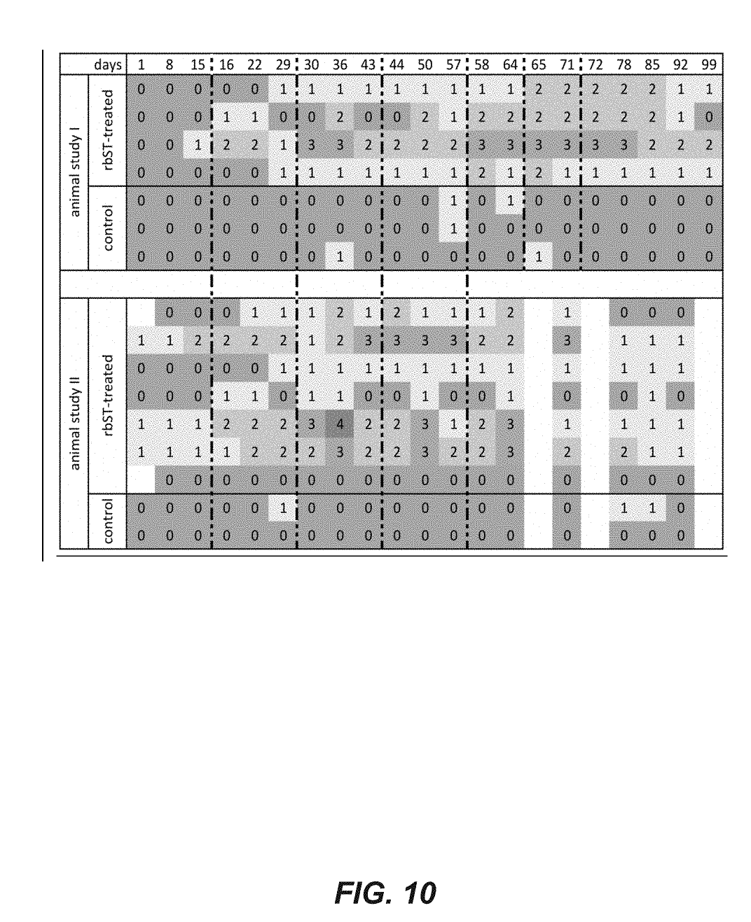

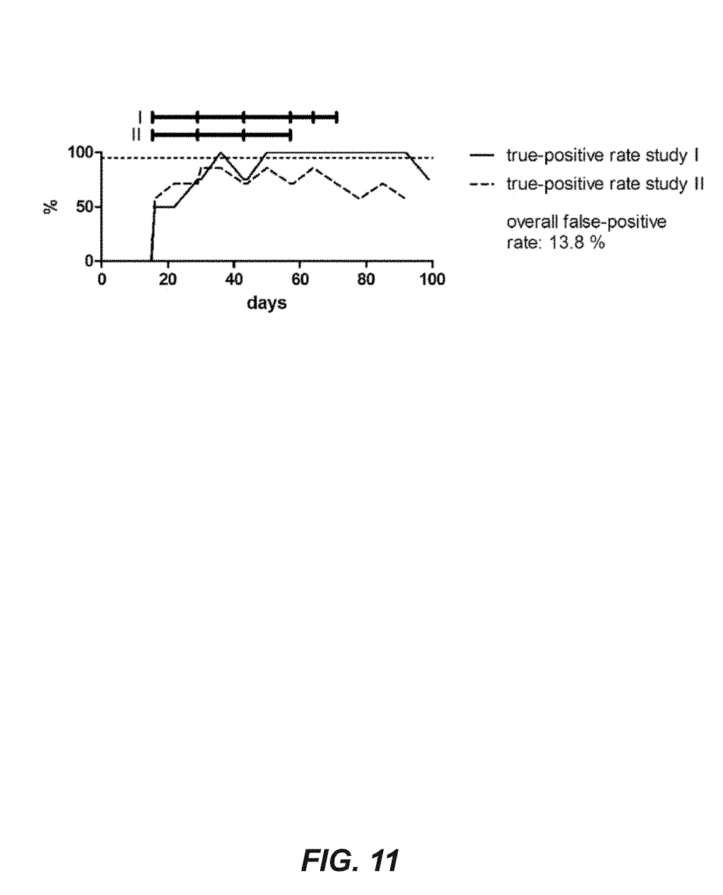

Since no single candidate biomarker was capable of predicting 95% of the rbST-treated samples correctly, different possibilities of combining biomarker results for improvement of the predictive power of the four-plex FCIA were tested. One approach to do this is the additive biomarker analysis. In FIG. 10, the number of candidate biomarkers responding above decision limit per cow and per time point within the animal studies is shown. As already described herein, there were big inter-individual differences: some cows responded in many markers, others only in one or two for some time points. There were also two extreme cases: one cow responded in all four tested markers above decision limit at one time point and another rbST-treated cow did not show any response above decision limit at any day. On the other hand, there were untreated cows, which showed positive responses in one of the candidate biomarkers. FIG. 11 shows the true-positive rate obtained for the rbST-treated cows of both animal studies considering a sample positive, when at least one biomarker reacted above the respective decision limit. Although the true-positive rate obtained with the additive biomarker analysis was much higher than for the single candidate biomarkers, the 95% true-positive rate required for a screening method was only reached at some time points in study I within the biweekly treatment period. After the double rbST injection in study I, all of the cows were found positive, but this treatment frequency will not be found in real practice. Furthermore, also with the additive biomarker analysis, quite some false-positive results were obtained throughout the whole experiment.

### Statistical Multiple Biomarker Analysis

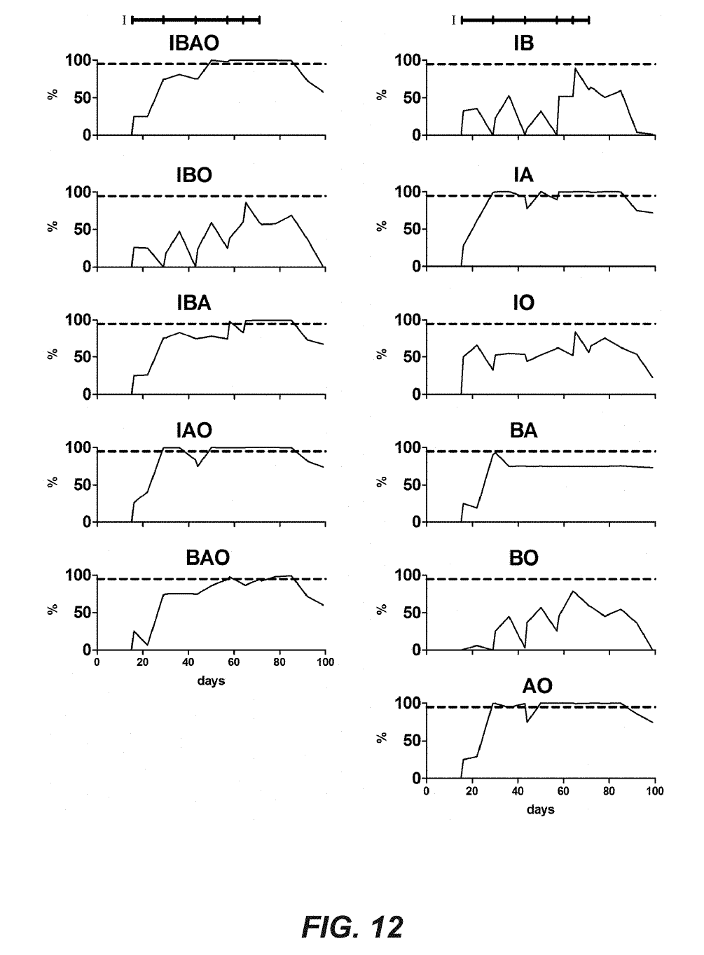

Since the single biomarker analysis and additive biomarker analysis, which were both based on decision limits, did not deliver satisfying results for predicting rbST abuse, a different biomarker-combining approach was chosen for analysis of the data. K-nearest neighbors (kNN), a statistical prediction tool, was used to build a model from one group of data (Group A: all animals of animal study II and untreated animals from animal study I) and predict the results of Group B (rbST-treated cows of animal study I and 67 independent untreated cows) on basis of the built model. Eleven different models (one for every possible combination of two to four biomarkers) were evaluated to find the optimal biomarker combination for rbST abuse prediction. True-positive rates of Group B data were calculated for every biomarker combination over the time of the whole animal study and are shown in FIG. 12. Six of the eleven different models yielded true-positive rates above the 95% true-positive rate required for a screening method at several time points. For the biomarker combinations IGF-1-IGFBP2-anti-rbST-antibodies (IBA) and IGFBP2-anti-rbST-antibodies-osteocalcin (BAO), only one time point within the biweekly treatment period was above 95%. Note that in total, samples from eleven time points were obtained and analyzed during the biweekly treatment period of animal study 1. For the biomarker combinations IBAO and IA, four and six time points within the biweekly treatment period were above the 95% target, respectively. Seven time points above the 95% target within the biweekly treatment period were reached by the prediction models based on the biomarker combinations IAO (IGF-1-anti-rbST-antibodies-osteocalcin) and AO (anti-rbST-antibodies-osteocalcin). For the three best performing models (IA (IGF-1-anti-rbST-antibodies), AO and IAO), true-positive rates above 95% (<5% false-compliant) were reached following the second rbST injection. For IA, a true-prediction rate of almost 60% was observed already one week after the first rbST injection, whereas AO only showed 30%, which is in accordance with expectations since IGF-1 is a quick responding biomarker and osteocalcin has a delayed response time. Since all of the rbST-treated cows were detected by the three best performing models (IA, AO, IAO) at the end of the biweekly treatment period, no further increased prediction rate was observed due to the subsequent two weekly injections. After withdrawal of rbST, the true-positive rate of the models based on IA, AO and IAO remained above 95% for two more weeks and then declined to 70% four weeks after withdrawal.

Since all of the untreated animals of both animal studies for model building were used, false-positive rates for the eleven different models were calculated based on the results of the 67 independent untreated cows (Table 4). For the three best-performing prediction models IA, AO and IAO, false-positive rates ranged from 10.6% to 14.7%, which was quite acceptable, since samples screened positive must be analyzed by a subsequent confirmatory analysis method according to Commission Decision 2002/657/EC anyway (European Union (2002) Commission Decision 2002/657/EC, Off. J. Eur. Commun. L. 221: 8-36). The confirmation method is based on the detection of an N-terminal peptide of somatotropin, which has a different terminal amino acid in the recombinant form of the hormone (Le Breton et al., 2010, J. Agr. Food Chem. 58:729-733).

It was concluded from the results of the studies presented herein that the (anti-rbST-antibodies-osteocalcin (AO) biomarker combination is the preferred model for predicting rbST abuse. It yielded seven out of eleven time points above the 95% target and if two biomarkers are equally well-suited for prediction as three biomarkers, the simpler version is favored

TABLE 4
False-positive rates of the statistical multiple biomarker analysis.
Results were calculated for 67 independent untreated cows predicted
with the eleven different biomarker combination models.

| | biomarker combination | | | | | | | | | | |
|---|---|---|---|---|---|---|---|---|---|---|---|
| | IBAO | IBO | IBA | IAO | BAO | IB | IA | IO | BA | BO | AO |
| | false-positive rate [%] | | | | | | | | | | |
| 67 untreated cows | 5.5 | 9.7 | 6.6 | 10.6 | 8.8 | 25 | 14.7 | 24.6 | 13.4 | 11.7 | 11.8 |

The results obtained proof that the developed four-plex FCIA reduced to an AO biomarker combination two-plex FCIA, applied to an in vivo evaluation and combined with a thorough statistical multiple biomarker analysis can detect more than 95% of the rbST-treated cows truly positive directly after the second rbST injection until the end of their treatment period and even thereafter. This meets the requirements of Commission Decision 2002/657/EC for a screening assay for the detection of banned veterinary drugs such as rbST ((European Union (2002) Commission Decision 2002/657/EC, Off. J. Eur. Commun. L. 221:8-36).

When comparing with previously reported results of a three-plex FCIA combining IGF-1, IGFBP2 and anti-rbST-antibodies (Smits et al., 2013, Analyst 138:111-117), the models presented here seemingly perform somewhat less, especially at the beginning of the rbST treatment but the new models are much more realistic: Note that here, two completely independent groups were used for model building (Group A) and prediction (Group B), whereas in the three-plex experiments (Smits et al., 2013, Analyst 138:111-117), sample data used for prediction were from the same cows as the data on which the model was built, leading to an overestimation of true-positive results in that work.

What is claimed is:

1. A method for determining whether a subject has been treated with exogenous somatotropin, the method comprising
determining a level of somatotropin in a sample from the subject;
comparing the determined level of somatotropin with a level of somatotropin in a reference; and
determining whether the subject has been treated with exogenous somatotropin if the level of somatotropin is increased in the sample of the subject, when compared to the reference.

2. The method according to claim 1, wherein the biological sample is a bodily fluid.

3. The method according to claim 1, wherein the subject is a bovine.

4. The method according to claim 3, wherein the bovine is a cow.

5. The method according to claim 1, wherein a level of bovine somatotropin is determined with the aid of an antibody or a functional part thereof directed against bovine somatotropin, followed by mass spectrometry.

6. The method according to claim 1, wherein the exogenous somatotropin is recombinant bovine somatotropin and a level of recombinant bovine somatotropin is determined utilizing an antibody or a functional part thereof directed against recombinant bovine somatotropin.

7. The method according to claim 1, wherein somatotropin is concentrated by affinity chromatography.

8. The method according to claim 1, wherein somatotropin is concentrated by repeated incubation with beads or monolithic material comprising affinity partners for somatotropin under circumstances that allow binding of somatotropin to the affinity partners.

9. The method according to claim 1, wherein somatotropin is digested.

10. The method according to claim 6, wherein said antibody directed against recombinant bovine somatotropin is generated by the subject.

11. The method according to claim 1, further comprising
determining a level of expression of at least one somatotropin-responsive gene product in said sample from the subject;
comparing the determined level of expression of said at least one somatotropin-responsive gene product with a level of expression of said at least one somatotropin-responsive gene product in a reference; and
determining whether a subject has been treated with exogenous somatotropin if the level of expression of said at least one somatotropin-responsive gene product differs from the level of expression as determined in said reference.

12. The method according to claim 11, wherein said at least one somatotropin-responsive gene product is selected from insulin-like growth factor 1 (IGF1), anti-rbST-antibodies, osteocalcin, and/or IGFBP2.

13. The method according to claim 11, wherein said at least one somatotropin-responsive gene product comprises insulin-like growth factor 1 (IGF1).

14. The method according to claim 11, wherein said at least one somatotropin-responsive gene product comprises insulin-like growth factor 1 (IGF1) and anti-rbST-antibodies.

15. The method according to claim 1, wherein said level of somatotropin is determined by Enzyme-Linked Immuno-Sorbent Assay (ELISA) or flow cytometric immunoassay (FCIA).

16. The method according to claim 2, wherein the bodily fluid is blood or milk.

17. The method according to claim 9, wherein somatotropin is digested with trypsin.

18. The method according to claim 17, wherein somatotropin is digested with trypsin for a period of between 0.2 and 5 hours.
